(19) United States
(12) Patent Application Publication
(10) Pub. No.: US 2022/0328128 A1
(43) Pub. Date: Oct. 13, 2022

(54) DOWNSCALING PARAMETERS TO DESIGN EXPERIMENTS AND PLATE MODELS FOR MICRO-ORGANISMS AT SMALL SCALE TO IMPROVE PREDICTION OF PERFORMANCE AT LARGER SCALE

(71) Applicant: Zymergen Inc., Emeryville, CA (US)

(72) Inventors: Stefan De Kok, Berkeley, CA (US); Peter Enyeart, Emeryville, CA (US); Richard Hansen, San Carlos, CA (US); Trent Hauck, Seattle, WA (US); Crystal Humphries, Kirkland, WA (US); Sarah Lieder, Oakland, CA (US); Zachariah Serber, Kenwood, CA (US); Erin Shellman, Seattle, WA (US); Amelia Taylor, Bend, OR (US); Thomas Treynor, Berkeley, CA (US); Kristina Tyner, Richmond, CA (US)

(73) Assignee: Zymergen Inc., Emeryville, CA (US)

(21) Appl. No.: 17/608,871

(22) PCT Filed: May 5, 2020
(86) PCT No.: PCT/US20/31482

§ 371 Date: Nov. 4, 2021

Related U.S. Application Data
(60) Provisional application No. 62/844,975, filed on May 8, 2019.

(51) Int. Cl. G16B 20/00; G16B 40/20; C12Q 1/02; G06Q 10/06; G05B 13/02; G06N 20/00

(52) U.S. Cl. CPC G16B 20/00; G16B 40/20; C12Q 1/02; G06Q 10/06393; G05B 13/024; G06N 20/00

(57) ABSTRACT

Systems, methods and computer-readable media are provided for designing experiments for organisms at a first scale to generate first-scale performance data used in predicting performance of the organisms at a second, larger scale. The design includes determining first-scale screening conditions based at least in part upon the contribution of second-scale conditions to performance parameters of an organism at the second scale. The first-scale screening conditions include one or more proxies for second-scale conditions that cannot be replicated at first scale. The design determines first-scale screening parameters based at least in part upon computer modeling of the metabolism of the organism at the second scale.

### CROSS-REFERENCE TO RELATED APPLICATIONS

This application claims the benefit of priority of U.S. Provisional Application No. 62/844,975, filed May 8, 2019. This application is related to: International Application No. PCT/US18/60120 (Pub. No. WO 2019/094787), filed on Nov. 9, 2018 (the “Transfer Function application”), which claims the benefit of priority to U.S. Provisional Application No. 62/583,961, filed Nov. 9, 2017; International Application No. PCT/US2017/029725 (U.S. Patent Pub. No. US 2017/0316353), filed on Apr. 26, 2017 (the “Codon application”), which claims the benefit of priority to U.S. application Ser. No. 15/140,296, filed on Apr. 27, 2016; U.S. Pat. No. 9,988,624 (the “HTP patent”); and International Application No. PCT/US2018/057583 (Pub. No. WO/2019/084315), which claims priority to U.S. Application No. 62/577,615, filed Oct. 26, 2017. All of the foregoing are hereby incorporated by reference herein in their entirety.

### BACKGROUND

### Field of the Disclosure

The present disclosure is generally directed to high-throughput genomic engineering of microorganisms, and, more particularly, to designing experiments for microorganisms at a first (e.g., plate) scale to support modeling of performance of the organisms at a second, larger scale, in order to enable efficient screening of the organisms at the first scale.

### Description of Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Microbe engineering enables the generation of novel chemicals, advanced materials, and pharmaceuticals. A strain design company, on behalf of itself or third parties, may modify a previously described DNA segment to enhance the metabolic production of a microbial host by improving output properties such as yield, productivity, growth rate, and titer.

One approach to optimizing the performance of an incompletely understood system, such as a living cell, is to test as many different genetic modifications as possible and empirically determine which perform best. Since testing modifications at a scale relevant to industrial production is typically expensive and time-consuming, the throughput for testing modifications at scale is very low. Therefore, the assignee of the present disclosure conducts small-scale, high-throughput screening to quickly identify the best candidates for performance from among large numbers of modifications. For this approach to be successful, however, there must be a reliable means of predicting larger-scale performance from smaller-scale performance. As examples, the scales range from small plates with many wells (e.g., 200-μL per well), to larger plates with fewer wells, to bench-scale tanks (e.g., 200 ml-10 liters), to commercial/industrial-sized tanks (e.g., 100-500,000 liters).

A technical field where such approaches have been widely applied is in the pharmaceutical industry, for purposes of identifying new and useful drugs. Thousands of candidate molecules may be first screened in vitro for activity in an assay that is expected to be a predictive proxy for in vivo activity. Statistical approaches are applied to determine the best performers (see, for example, Malo et al. “Statistical practice in high-throughput screening data analysis.” Nat Biotechnol 24:167-175 (2006)), which are then used in more expensive, larger scale experiments, which may include in vivo testing in mice and humans.

However, when screening many thousands of microorganisms for desired properties, the efficient determination of reasonably promising performance parameters and conditions to use in screening at the plate level becomes critical to enable reliable prediction at larger scale.

Delvigne, 2017 summarizes the academic proposed solution, progress on those solutions and thoughts on scale-up challenges as follows: “The bio-economy is in transit from innovation to commercialization. The bioprocess industry is expected to increasingly deliver bio-products to the market, in large amounts, at high quality and at competitive cost levels. This requires flawless start-up of new large-scale bioprocesses and continuous improvement of running processes. Fermentation scale-up and operation can benefit from recent advances in three areas: 1. computation-driven design of scale-down simulators, 2. omics-driven metabolic engineering and, 3. sensing and understanding of population heterogeneity. Integration of these fields requires a unified computational approach, linked to big data and simulated reality frameworks, of which the contours are becoming visible today.” F. Delvigne, et al., Scale-up/Scale-down of microbial bioprocesses: a modern light on an old issue, Microb. Biotechnol. 2017 July; 10(4):685-687.

Driving Innovation Through Bioengineering Solutions, Genomatica (date unknown) (“Genomatica”) describes designing lab-scale “scale-down” experiments that de-risk the scale-up to commercial scale. Genomatica describes developing predictive models of commercial-scale fermenters, and linking the microbe's metabolism to reactor design, and optimizing microbe and fermentation processes under large-scale conditions. However, Genomatica does not teach high-throughput screening or developing experiments at plate scale, and thus does not recognize the challenges in developing screening conditions for plates.

### SUMMARY OF THE DISCLOSURE

In order to test many thousands of strain variants, the assignee employs a factory process that perform many thousands of small-scale experiments that are predictive of strain variant performance at a larger scale. A plate model is a manifestation of the factory process that enables the rapid testing of thousands of strain variants. Developing a plate model is a delicate balance of scaling down a larger scale process with optimizing a manufacturing process for the larger scale.

Embodiments of the disclosure scale down (and subsequently scale up) bioprocesses using a structured and analytical method to analyze large-scale (e.g., production scale, bench scale) fermentation processes, and scale down directly from large scale into high throughput screening of 96-well plates. According to embodiments of the disclosure, this approach is based on understanding the key driving parameters of the key performance indicator (“KPI”) through a thorough characterization of the fermentation process. Embodiments of the disclosure quantify the impact of various factors influencing performance of a microorganism using analytics and modelling of the performance measure and its interaction with the changing environment in the bioprocess.

Embodiments of the disclosure enable screening of thousands of strains in 96-well titer plates with an expected positive predictive value of >0.33 comparing plates with bench scale performance. Selected hits transferred successfully up to commercial scale, showing the success of predicting performance prediction from microliter scale to multiple hundred cubic meter commercial scale.

Embodiments of the disclosure design experiments and develop physical plate models, which are sets of experimental conditions and protocols used as inputs to a transfer function to model larger-scale (e.g., bench-scale tank) performance.

Embodiments of the disclosure employ multi-objective optimization (“MOO”) to decrease analysis time and increase the efficiency of plate model development. According to embodiments of the disclosure, MOO may be implemented using Response Surface Methodology (“RSM”), and may employ a metric, plate-tank deviance, to quickly sift through experimental condition parameters (e.g, media composition, inoculation volume) and their values to optimize the plate model for operations. Embodiments of the disclosure use a standardized quantifiable approach that optimizes measures of the organism's physiology (e.g., pH, glucose, biomass) and accounts for the need to have a plate-scale assay that is a proxy for yield and productivity in tanks. Further, it supports parameter interpolation, for more quantitative and faster decision-making. Using the embodiments of the disclosure reduces individual contributor's time and standardizes the process, while creating a plate model (scaled-down process) that performs well across multiple physiological and product production goals.

Embodiments of the disclosure design a preliminary plate model and experiments with the goal of finding the optimal values of process parameters like inoculation volume and plate types, cultivation conditions like temperature and target shake times and media components, among others, to use in operations. The core method used is an analytical framework that combines sequential experimental design statistical models and an optimization function to explore the relationship between multiple experimental parameters and one or more responses.

Embodiments of the disclosure provide systems, methods and computer-readable media storing instructions for designing experiments for organisms at a first scale to generate first-scale performance data used in predicting performance of the organisms at a larger, second scale. Embodiments of the disclosure:
determine first (e.g., plate) scale screening conditions based at least in part upon the contribution of second (e.g., bench) scale conditions to performance parameters of first strains of an organism at the second scale, wherein the first-scale screening conditions include one or more proxies for second-scale conditions that cannot be replicated at first scale;
determine first-scale screening parameters based at least in part upon computer modeling of the metabolism of the organism at the second scale; and
design experiments for experimentally screening second strains of the organism (which may, in embodiments, be the same as the first strains) under the first-scale screening conditions based at least in part upon the first-scale screening parameters.

According to embodiments of the disclosure, the first scale is at the scale of a plate comprising wells wherein each well has a volume within a range of 50-200 microliters, and the second scale is at the scale of a bench tank has a volume within a range of 200 ml-10 liters.

Embodiments of the disclosure generate a first-scale statistical model of first-scale performance of the second strains, and use the first-scale statistical model to predict performance of the second strains at a third scale (e.g., using the transfer function described herein). According to embodiments of the disclosure, the third scale is larger than the first and second scales. Alternatively, the third scale may be the same as the second scale. According to embodiments of the disclosure, designing experiments includes screening the second strains based at least in part upon the predicted third-scale performance of the second strains.

According to embodiments of the disclosure, determining first-scale screening conditions may also be based at least in part upon environmental conditions determined from fermentation modeling (e.g., of the organism at a third scale larger than the second scale).

According to embodiments of the disclosure, determining first-scale screening parameters comprises determining second-scale performance parameters that contribute to a key performance indicator (“KPI”) above a contribution threshold. According to embodiments of the disclosure, determining first-scale screening parameters comprises determining second-scale performance parameters based on their potential for improving performance of a KPI.

Embodiments of the disclosure determine optimum values of the first-scale screening conditions that optimize the first-scale screening parameters (and, in some embodiments, a plate-tank deviance) collectively at the first scale (e.g., using multi-objective optimization), and designing experiments comprises designing experiments to experimentally determine first-scale performance of the second strains in response to a range of screening condition values around the optimum screening condition values.

Embodiments of the disclosure control the performance of experiments to screen the second strains at the first scale using the first-scale screening conditions and the first scale screening parameters.

### BRIEF DESCRIPTION OF THE DRAWINGS

FIG. 1A is a system diagram of a laboratory information management system (LIMS) of embodiments of the disclosure for the high-throughput (“HTP”) design, building, testing, and analysis of DNA sequences.

FIG. 1B illustrates a distributed system of embodiments of the disclosure.

FIG. 1C and FIG. 1D are corresponding flow diagrams for LIMS.

FIG. 2A illustrates a comparison of measured bioreactor (tank, larger scale) vs. plate (smaller scale) values for individual strains, according to embodiments of the disclosure.

FIG. 2B illustrates a comparison of actual tank yield values to linear predicted tank yield values for a bioreactor (tank) in an example according to embodiments of the disclosure.

FIG. 3 is a plot equivalent to that of FIG. 2B, except with Type 1 outlier strain N removed.

FIG. 4 is a plot equivalent to that of FIG. 2B, except with four Type 1 outliers and one Type 2 outlier removed.

FIG. 5 depicts the result of applying a correction to all the strains in FIG. 4 based on whether or not they have a certain genetic modification, according to embodiments of the disclosure.

FIG. 6 is a regression plot of the model shown in FIG. 5, according to embodiments of the disclosure.

FIG. 7 illustrates a productivity model without correction for genetic factors, according to embodiments of the disclosure.

FIG. 8 illustrates the productivity model of FIG. 7 after correction for a genetic factor, according to embodiments of the disclosure.

FIG. 9 illustrates improvement in the high-throughput productivity-model performance (x-axis) versus improvement in actual productivity in low-throughput bioreactors (e.g., tanks) (y-axis) for strains harboring the same promoter swap as in FIG. 8.

FIG. 10 illustrates a user interface of a transfer function development tool according to embodiments of the disclosure.

FIG. 11 illustrates the user interface, according to embodiments of the disclosure.

FIG. 12 illustrates a user interface displaying a plate-tank correlation transfer function, according to embodiments of the disclosure.

FIG. 13 illustrates the user interface presenting ten strains having the highest predicted performance based upon the transfer function with the outliers selected by the user having been removed from the model, according to embodiments of the disclosure.

FIG. 14 illustrates a graphical representation of the chosen transfer function after user-selected outliers have been removed from the model, according to embodiments of the disclosure.

FIG. 15 illustrates an interface enabling the user to to submit quality scores for the removed strains to a database, according to embodiments of the disclosure.

FIG. 16 illustrates a cloud computing environment according to embodiments of the disclosure.

FIG. 17 illustrates an example of a computer system that may be used to execute program code to implement embodiments of the disclosure.

FIG. 18 is a graph of plate vs. tank values resulting from an experiment performed according to embodiments of the disclosure.

FIG. 19 is a graph of plate vs. tank values resulting from an experiment performed according to embodiments of the disclosure.

FIG. 20 is a graph of plate vs. tank values resulting from an experiment performed according to embodiments of the disclosure.

FIG. 21 is a graph of plate vs. tank values resulting from an experiment performed according to embodiments of the disclosure.

FIG. 22 is a graph of plate vs. tank values resulting from an experiment performed according to embodiments of the disclosure.

FIG. 23 is a graph of observed tank values vs. predicted tank values resulting from an experiment performed according to embodiments of the disclosure.

FIG. 24 is a graph of observed tank values vs. predicted tank values resulting from an experiment performed according to embodiments of the disclosure.

FIG. 25 is a graph plotting a first tank value vs. a second tank value resulting from an experiment performed according to embodiments of the disclosure.

FIG. 26 is a a graph of observed tank values vs. predicted tank values resulting from an experiment performed according to embodiments of the disclosure.

FIG. 27 plots sugar (Cs), product (Cp) and biomass (Cx) concentrations that were estimated over time according to a prophetic example based on embodiments of the disclosure.

FIG. 28 is a graph of product concentration vs. fermenter product yield according to a prophetic example based on embodiments of the disclosure.

FIG. 29 is a graph of sugar concentration vs. fermenter product yield according to a prophetic example based on embodiments of the disclosure.

FIG. 30 is a graph of biomass concentration vs. fermenter product yield according to a prophetic example based on embodiments of the disclosure.

FIG. 31 is a graph of product yield in plates vs. fermenter product yield according to a prophetic example based on embodiments of the disclosure.

FIGS. 32A and 32B illustrate steps for designing experiments for organisms at a first (plate) scale to generate first-scale performance data used in predicting performance of the organisms at a larger (e.g., bench or commercial) scale, according to embodiments of the disclosure.

FIG. 32C illustrates an RSM workflow for multi-objective optimization, according to embodiments of the disclosure.

FIG. 33 plots an example of accumulated titer measured over the course of a bioprocess at different elapsed fermentation times, according to embodiments of the disclosure.

according to embodiments of the disclosure.

FIG. 34 illustrates an example of a surface shape showing how biomass is modeled, according to embodiments of the disclosure.

FIGS. 35A and 35B depict steps for DNA assembly, transformation, and strain screening, according to embodiments of the disclosure

FIGS. 36A and 36B provide another view of high-throughput strain engineering, according to embodiments of the disclosure.

FIG. 37 illustrates an automated system of embodiments of the disclosure comprising work modules.

### DETAILED DESCRIPTION

The present description is made with reference to the accompanying drawings, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

As used herein the terms “organism” “microorganism” or “microbe” should be taken broadly. These terms are used interchangeably and include, but are not limited to, the two prokaryotic domains, Bacteria and Archaea, as well as certain eukaryotic fungi and protists.

A “high-throughput (HTP)” method of genomic engineering may involve the utilization of at least one piece of automated equipment (e.g. a liquid handler or plate handler machine) to carry out at least one step of said method.

Genomic Automation

Automation of the methods of the disclosure enables high-throughput phenotypic screening and identification of target products from multiple test strain variants simultaneously. Hundreds or thousands of mutant strains are constructed in a high-throughput fashion. The robotic and computer systems described below are the structural mechanisms by which such a high-throughput process can be carried out.

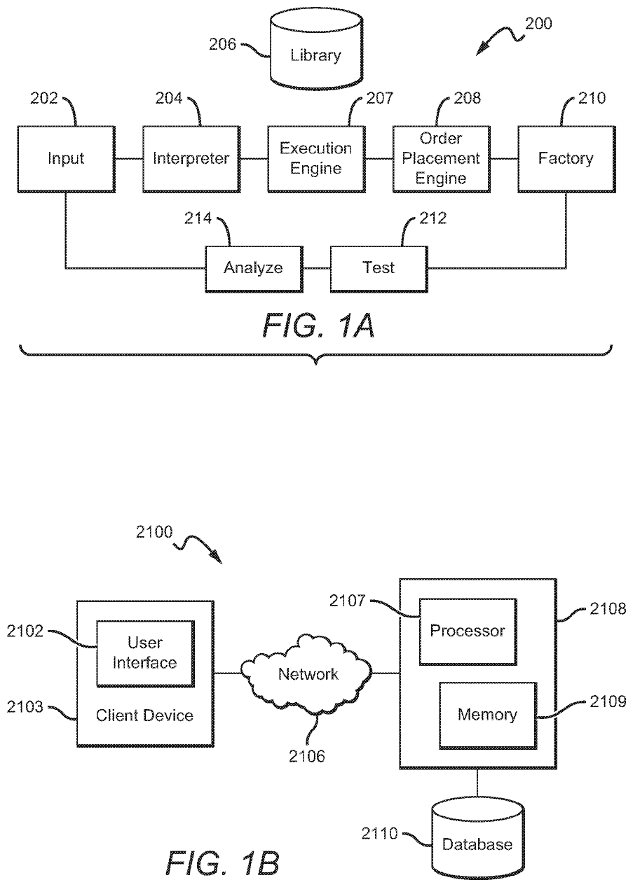

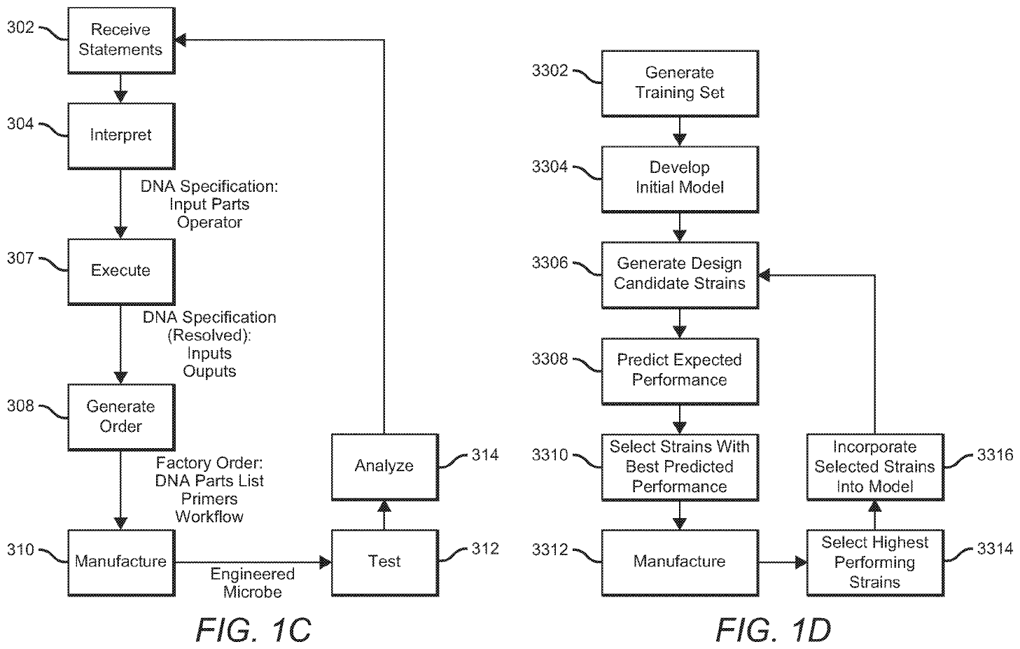

FIG. 1A is a system diagram of a laboratory information management system (LIMS) 200 of embodiments of the disclosure for the high-throughput (“HTP”) design, building, testing, and analysis of DNA sequences.

FIG. 1B illustrates a distributed system 2100 of embodiments of the disclosure. A user interface 2102 includes a client-side interface such as a text editor or a graphical user interface (GUI). The user interface 2102 may reside at a client-side computing device 2103, such as a laptop or desktop computer. The client-side computing device 2103 is coupled to one or more servers 2108 through a network 2106, such as the Internet.

The server(s) 2108 are coupled locally or remotely to one or more databases 2110, which may include one or more corpora of libraries including data such as genome data, genetic modification data (e.g., promoter ladders), process condition data, strain environmental data, and phenotypic performance data that may represent microbial strain performance at both small and large scales, and in response to genetic modifications. “Microbes” herein includes bacteria, fungi, and yeast.

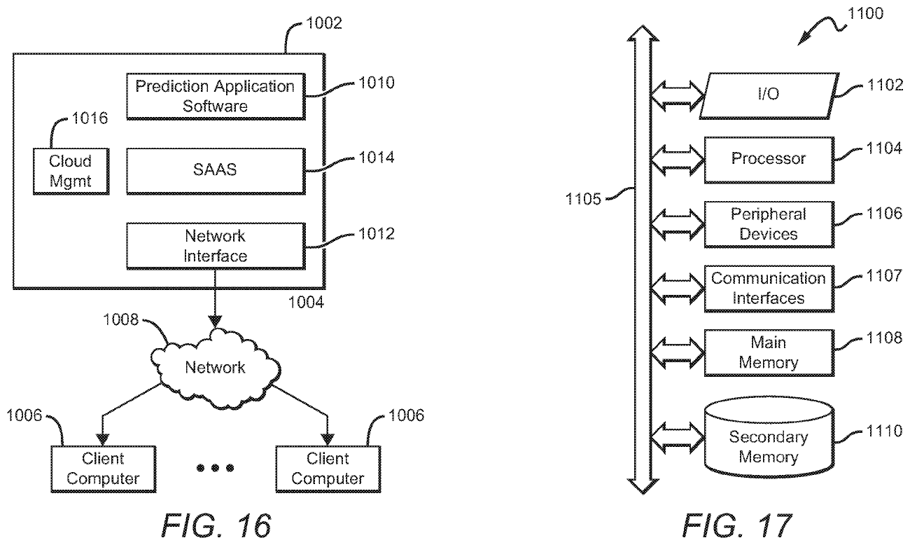

In embodiments, the server(s) 2108 include at least one processor 2107 and at least one memory 109 storing instructions that, when executed by the processor(s) 2107, perform operations disclosed herein, including generating a prediction function, thereby acting as a prediction engine according to embodiments of the disclosure. The same arrangement may act as the PM engine, the analysis equipment 214 or other elements of the LIMS system, or other computing elements, according to embodiments of the disclosure. Alternatively, the software and associated hardware for the these computing elements may reside locally at the client 2103 instead of at the server(s) 2108, or be distributed between both client 2103 and server(s) 2108. In embodiments, all or parts of the these computing elements may run as a cloud-based service, depicted further in FIG. 16. Note that the prediction engine and the PM engine may reside at the analysis equipment 214 of the LIMS.

The database(s) 2110 may include public databases, as well as custom databases generated by the user or others, e.g., databases including molecules generated via fermentation experiments performed by the user or third-party contributors. The database(s) 2110 may be local or remote with respect to the client 2103 or distributed both locally and remotely.

FIG. 1C and FIG. 1D are corresponding flow diagrams for LIMS 200. In embodiments of LIMS, many changes may be made to an input DNA sequence at a time, resulting in a single output sequence for each change or change set. To optimize strains (e.g., manufacture microbes that efficiently produce an organic compound with high yield), LIMS produces many such DNA output sequences at a time, so that they may be analyzed within the same timeframe to determine which host cells, and thus which modifications to the input sequence, best achieve the desired properties.

In some embodiments the system enables the design of multiple nucleotide sequence constructs (such as DNA constructs like promoters, codons, or genes), each with one or more changes, and creates a work order (i.e., “factory order”) to instruct a gene manufacturing system, factory 210, to build the nucleotide sequence constructs in the form of microbes carrying the constructs. Examples of microbes that may be built include, without limitation, hosts such as bacteria, fungi, and yeast. According to the system, the microbes are then tested for their properties (e.g., yield, titer). In feedback-loop fashion, the results are analyzed to iteratively improve upon the designs of prior generations to achieve more optimal microbe performance.

Although the design, build, test and analysis process is described herein primarily in the context of microbial genome modification, those skilled in the art will recognize that this process may be used for desired gene modification and expression goals in any type of host cell.

Referring to FIGS. 1A-1D in more detail, an input interface 1202, such as a computer running a program editor, receives statements of a program/script that is used to design one or more DNA output sequences (see 302). Such a genomic design program language may be referred to herein as the “Codon” programming language developed by the assignee of the present disclosure, and described herein in the Codon application reference above. A powerful feature of embodiments of the disclosure is the ability to develop designs for a very large number of DNA sequences (e.g., microbial strains, plasmids) within the same program with just a few procedural statements.

Here, the editor enables a user to enter and edit the program, e.g., through graphical or text entry or via menus or forms using a keyboard and mouse on a computing device. Those skilled in the art will recognize that other input interfaces 202 may be employed without the need for direct user input, e.g., the input interface 202 may employ an application programming interface (API), and receive statements in files comprising the program from another computing device. The input interface 202 may communicate with other elements of the system over local or remote connections.

As described in the Codon application, an interpreter or compiler/execution unit 204 evaluates program statements into novel DNA specification data structures of embodiments of the disclosure (304). According to embodiments of the disclosure, the interpreter 204, along with the execution engine 207 and the order placement engine 208 transforms the progam statements from a logical specification into a specification of a physical manufacturing process for use by the factory 210.

The factory order placer 208 can determine the intermediate parts that will be required for that workflow process performed by the factory 210 using libraries of known parameters and known algorithms that obey known heuristics and other properties (e.g., optimal melting temperature to run on common equipment).

The resulting factory order may include a combination of a prescribed set of steps, as well as the parameters, inputs and outputs for each of those steps for each DNA sequence to be constructed. The factory order may include a DNA parts list including a starting microbial base strain, a list of primers, guide RNA sequences, or other template components or reagent specifications necessary to effect the workflow, along with one or more manufacturing workflow specifications for different operations within the DNA specification. These primary, intermediate, and final parts or strains may be reified via a factory build graph; the workflow steps refer to elements of the build graph with various roles. The order placement engine 208 may refer to the library 206 for the information discussed above. According to embodiments of the disclosure, this information is used to reify the design campaign operations in physical (as opposed to in silico) form at the factory 210 based upon conventional techniques for nucleotide sequence synthesis, as well as custom techniques developed by users or others.

For example, assume a recursive program statement has a top-level function of circularize and its input is a chain of concatenate specifications. The factory order placer 208 may interpret that series of inputs such that a person or robot in the lab may perform a PCR reaction to amplify each of the inputs and then assemble them into a circular plasmid, according to conventional techniques or custom/improved techniques developed by the user. The factory order may specify the PCR products that should be created in order to do the assembly. The factory order may also provide the primers that should be purchased in order to perform the PCR.

In another example, assume a program statement specifies a top-level function of replace. The factory order placer 208 may interpret this as a cell transformation (a process that replaces one section of a genome with another in a live cell). Furthermore, the inputs to the replace function may include parameters that indicate the source of the DNA (e.g. cut out of another plasmid, amplified off some other strain).

The order placement engine 208 may communicate the factory order to the factory 210 over local or remote connections. Based upon the factory order, the factory 210 may acquire short DNA parts from outside vendors and internal storage, and employ techniques known in the art, such as the Gibson assembly protocol or the Golden Gate Assembly protocol, to assemble DNA sequences corresponding to the input designs (310). The factory order itself may specify which techniques to employ during beginning, intermediate and final stages of manufacture. For example, many laboratory protocols include a PCR amplification step that requires a template sequence and two primer sequences. The factory 210 may be implemented partially or wholly using robotic automation.

According to embodiments of the disclosure, the factory order may specify the production in the factory 210 of hundreds or thousands of DNA constructs, each with a different genetic makeup. The DNA constructs are typically circularized to form plasmids for insertion into the base strain. In the factory 210, the base strain is prepared to receive the assembled plasmid, which is then inserted.

The resulting DNA sequences assembled at the factory 210 are tested using test equipment 212 (312). During testing, the microbe strains are subjected to quality control (QC) assessments based upon size and sequencing methods. The resulting, modified strains that pass QC may then be transferred from liquid or colony cultures on to plates. Under environmental conditions that model production conditions, the strains are grown and then assayed to test performance (e.g., desired product concentration). The same test process may be performed in flasks or tanks.

In feedback-loop fashion, the results may be analyzed by analysis equipment 214 to determine which microbes exhibit desired phenotypic properties (314). During the analysis phase, the modified strain cultures are evaluated to determine their performance, i.e., their expression of desired phenotypic properties, including the ability to be produced at industrial scale. The analysis phase uses, among other things, image data of plates to measure microbial colony growth as an indicator of colony health. The analysis equipment 214 may include a computer to perform a number of operations described herein, including correlating genetic changes with phenotypic performance, and saving the resulting genotype-phenotype correlation data in libraries, which may be stored in library 206, to inform future microbial production.

LIMS iterates the design/build/test/analyze cycle based on the correlations developed from previous factory runs. During a subsequent cycle, the analysis equipment 214, alone or in conjunction with human operators, may select the best candidates as base strains for input back into input interface 202, using the correlation data to fine tune genetic modifications to achieve better phenotypic performance with finer granularity. In this manner, the laboratory information management system of embodiments of the disclosure implements a quality improvement feedback loop.

Those skilled in the art will recognize that some embodiments described herein may be performed entirely through automated means of the LIMS system 200, e.g., by the analysis equipment 214, or by human implementation, or through a combination of automated and manual means. When an operation is not fully automated, the elements of the LIMS system 200, e.g., analysis equipment 214, may, for example, receive the results of the human performance of the operations rather than generate results through its own operational capabilities. As described elsewhere herein, components of the LIMS system 200, such as the analysis equipment 214, may be implemented wholly or partially by one or more computer systems. In some embodiments, in particular where operations are performed by a combination of automated and manual means, the analysis equipment 214 may include not only computer hardware, software or firmware (or a combination thereof), but also equipment operated by a human operator such as that listed in Table 1 below.

In some embodiments, the high-throughput screening process is designed to predict performance of strains in bioreactors. As previously described, culture conditions are selected to be suitable for the organism and reflective of bioreactor conditions. Individual colonies are picked and transferred into 96 well plates and incubated for a suitable amount of time. Cells are subsequently transferred to new 96 well plates for additional seed cultures, or to production cultures. Cultures are incubated for varying lengths of time, where multiple measurements may be made. These may include measurements of product, biomass or other characteristics that predict performance of strains in bioreactors. High-throughput culture results are used to predict bioreactor performance.

In some embodiments, the tank-based performance validation is used to confirm performance of strains isolated by high throughput screening. Fermentation processes/conditions may be obtained from customers of the operator of the LIMS system. Candidate strains may be screened using bench scale fermentation reactors (e.g., reactors disclosed in Table 1 of the present disclosure) for relevant strain performance characteristics such as productivity or yield.

Iterative Strain Design Optimization

Referring to FIGS. 1A-1C, the order placement engine 208 places a factory order to the factory 210 to manufacture microbial strains incorporating the candidate mutations, according to embodiments of the disclosure. In feedback-loop fashion, the results may be analyzed by the analysis equipment 214 to determine which microbes exhibit desired phenotypic properties (314). During the analysis phase, the modified strain cultures are evaluated to determine their performance, i.e., their expression of desired phenotypic properties, including the ability to be produced at industrial scale. For example, the analysis phase uses, among other things, image data of plates to measure microbial colony growth as an indicator of colony health. The analysis equipment 214 is used to correlate genetic changes with phenotypic performance, and save the resulting genotype-phenotype correlation data in libraries, which may be stored in library 206, to inform future microbial production.

In particular, the genotype-phenotype correlation data resulting from candidate changes that result in sufficiently high measured performance may be added to a training data set. In this manner, the best performing mutations are added to a predictive strain design model in a supervised machine learning fashion.

LIMS iterates the design/build/test/analyze cycle based on the correlations developed from previous factory runs. During a subsequent cycle, the analysis equipment 214 alone, or in conjunction with human operators, may select the best candidates as base strains for input back into input interface 202, using the correlation data to fine tune genetic modifications to achieve better phenotypic performance with finer granularity. In this manner, the laboratory information management system of embodiments of the disclosure implements a quality improvement feedback loop.

In sum, with reference to the flowchart of FIG. 1C the iterative predictive strain design workflow may be described as follows:
Generate a training set of input and output variables, e.g., genetic changes as inputs and performance features as outputs (3302). Generation may be performed by the analysis equipment 214 based upon previous genetic changes and the corresponding measured performance of the microbial strains incorporating those genetic changes.
Develop an initial model (e.g., linear regression model) based upon training set (3304). This may be performed by the analysis equipment 214.
Generate design candidate strains (3306)
   In one embodiment, the analysis equipment 214 may fix the number of genetic changes to be made to a background strain, in the form of combinations of changes. To represent these changes, the analysis equipment 214 may provide to the interpreter 204 one or more DNA specification expressions representing those combinations of changes. (These genetic changes or the microbial strains incorporating those changes may be referred to as “test inputs.”) The interpreter 204 interprets the one or more DNA specifications, and the execution engine 207 executes the DNA specifications to populate the DNA specification with resolved outputs representing the individual candidate design strains for those changes.
Based upon the model, the analysis equipment 214 predicts expected performance of each candidate design strain (3308).
The analysis equipment 214 selects a limited number of candidate designs, e.g., 100, with highest predicted performance (3310).
   The analysis equipment 214 may account for second-order effects such as epistasis, by, e.g., filtering top designs for epistatic effects, or factoring epistasis into the predictive model.
Build the filtered candidate strains (at the factory 210) based on the factory order generated by the order placement engine 208 (3312).
The analysis equipment 214 measures the actual performance of the selected strains, selects a limited number of those selected strains based upon their superior actual performance (3314), and adds the design changes and their resulting performance to the predictive model (3316). The predictive model may employ linear regression.
The analysis equipment 214 then iterates back to generation of new design candidate strains (3306), and continues iterating until a stop condition is satisfied. The stop condition may comprise, for example, the measured performance of at least one microbial strain satisfying a performance metric, such as yield, growth rate, or titer.

In the example above, the iterative optimization of strain design may employ feedback and linear regression to implement machine learning.

Other General HTP Descriptions

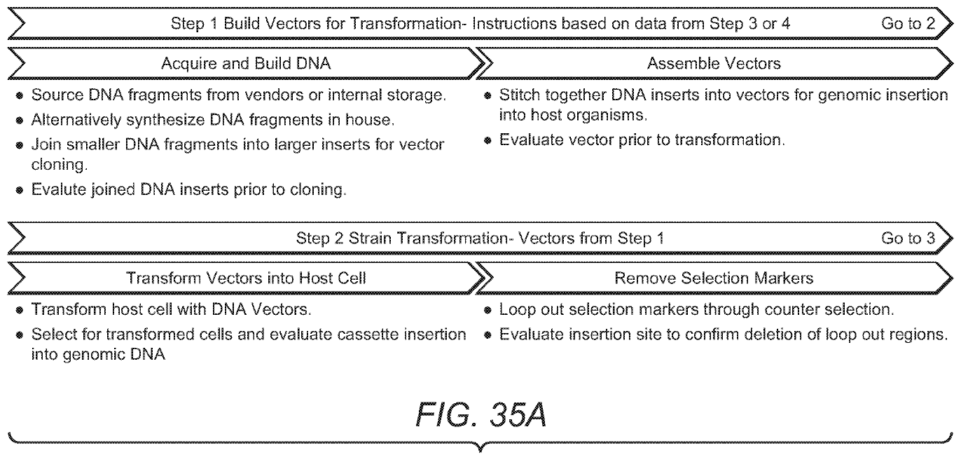

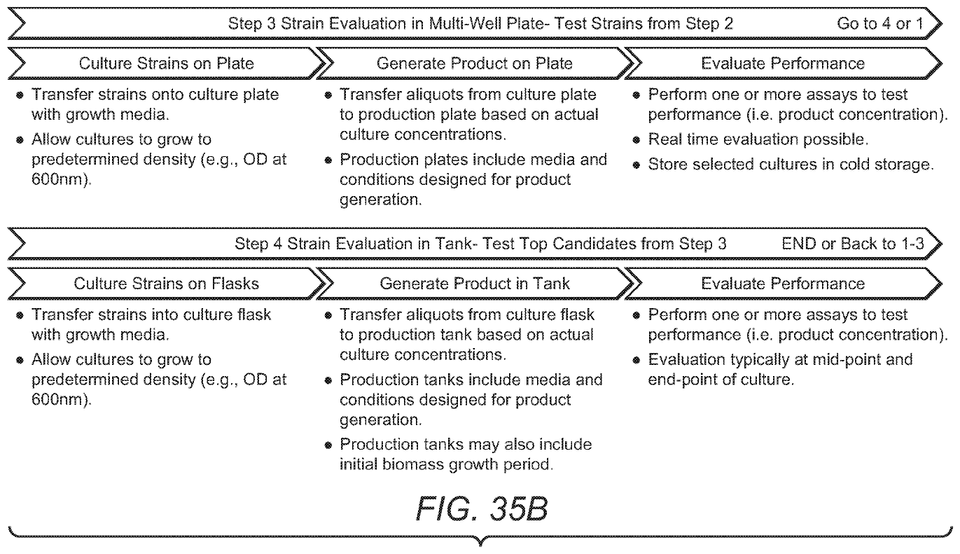

FIGS. 35A and 35B depict steps for DNA assembly, transformation, and strain screening, according to embodiments of the disclosure. FIG. 35A depicts steps for building DNA fragments, cloning DNA fragments into vectors, transforming the vectors into host strains, and removing selection markers. FIG. 35B depicts steps for high-throughput culturing, screening, and evaluation of selected host strains. This figure also depicts optional steps of culturing, screening, and evaluating selected strains in culture tanks.

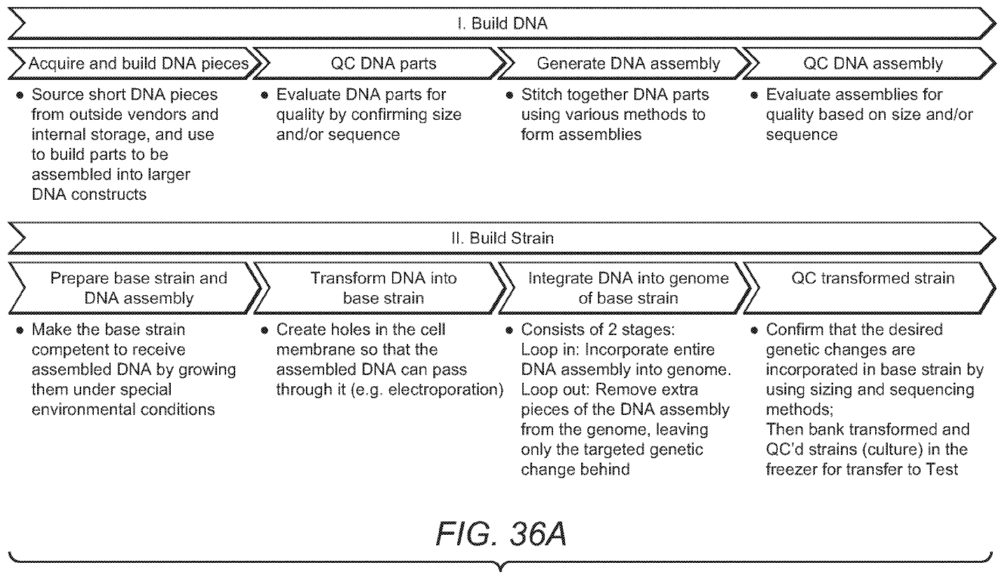

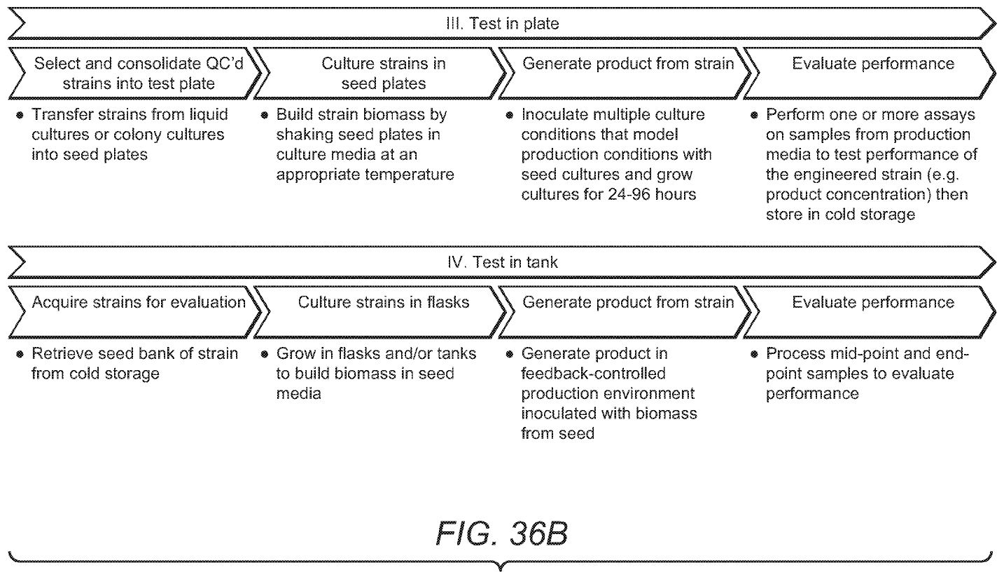

FIGS. 36A and 36B provide another view of high-throughput strain engineering, according to embodiments of the disclosure. The flow chart depicts steps for building DNA, building strains from the DNA, and testing strains in plates and in tanks.

HTP Robotic Systems

According to embodiments of the disclosure, the automated HTP methods of the disclosure comprise a robotic system. The systems outlined herein are generally directed to the use of 96- or 384-well microtiter plates, but as will be appreciated by those in the art, any number of different plates or configurations may be used. In addition, any or all of the steps outlined herein may be completely or partially automated.

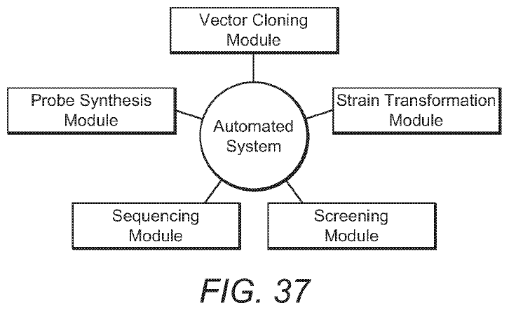

Referring to FIG. 37, automated systems of embodiments of the disclosure comprise one or more work modules. For example, in some embodiments, automated robotic systems system include a DNA synthesis module, a vector cloning module, a strain transformation module, a screening module, and a sequencing module capable of cloning, transforming, culturing, screening and sequencing host organisms.

As will be appreciated by those in the art, an automated system can include a wide variety of components, including, but not limited to: liquid handlers; one or more robotic arms; plate handlers for the positioning of microplates; plate sealers, plate piercers, automated lid handlers to remove and replace lids for wells on non-cross contamination plates; disposable tip assemblies for sample distribution with disposable tips; washable tip assemblies for sample distribution; 96 well loading blocks; integrated thermal cyclers; cooled reagent racks; microtiter plate pipette positions (optionally cooled); stacking towers for plates and tips; magnetic bead processing stations; filtrations systems; plate shakers; barcode readers and applicators; and computer systems.

In some embodiments, the robotic systems of the present disclosure include automated liquid and particle handling enabling high-throughput pipetting to perform all the steps in the process of gene targeting and recombination applications. This includes liquid and particle manipulations such as aspiration, dispensing, mixing, diluting, washing, accurate volumetric transfers; retrieving and discarding of pipette tips; and repetitive pipetting of identical volumes for multiple deliveries from a single sample aspiration. These manipulations are cross-contamination-free liquid, particle, cell, and organism transfers. The instruments perform automated replication of microplate samples to filters, membranes, and/or daughter plates, high-density transfers, full-plate serial dilutions, and high capacity operation.

In some embodiments, the customized automated liquid handling system of the disclosure is a TECAN machine (e.g. a customized TECAN Freedom Evo).

In some embodiments, the automated systems of the present disclosure are compatible with platforms for multi-well plates, deep-well plates, square well plates, reagent troughs, test tubes, mini tubes, microfuge tubes, cryovials, filters, micro array chips, optic fibers, beads, agarose and acrylamide gels, and other solid-phase matrices or platforms are accommodated on an upgradeable modular deck. In some embodiments, the automated systems of the present disclosure contain at least one modular deck for multi-position work surfaces for placing source and output samples, reagents, sample and reagent dilution, assay plates, sample and reagent reservoirs, pipette tips, and an active tip-washing station.

In some embodiments, the automated systems of the present disclosure include high-throughput electroporation systems. In some embodiments, the high-throughput electroporation systems are capable of transforming cells in 96 or 384-well plates. In some embodiments, the high-throughput electroporation systems include VWR® High-throughput Electroporation Systems, BTX™, Bio-Rad® Gene Pulser MXcell™ or other multi-well electroporation system.

In some embodiments, the integrated thermal cycler and/or thermal regulators are used for stabilizing the temperature of heat exchangers such as controlled blocks or platforms to provide accurate temperature control of incubating samples from 0° C. to 100° C.

In some embodiments, the automated systems of the present disclosure are compatible with interchangeable machine-heads (single or multi-channel) with single or multiple magnetic probes, affinity probes, replicators or pipetters, capable of robotically manipulating liquid, particles, cells, and multi-cellular organisms. Multi-well or multi-tube magnetic separators and filtration stations manipulate liquid, particles, cells, and organisms in single or multiple sample formats.

In some embodiments, the automated systems of the present disclosure are compatible with camera vision and/or spectrometer systems. Thus, in some embodiments, the automated systems of the present disclosure are capable of detecting and logging color and absorption changes in ongoing cellular cultures.

In some embodiments, the automated system of the present disclosure is designed to be flexible and adaptable with multiple hardware add-ons to allow the system to carry out multiple applications. The software program modules allow creation, modification, and running of methods. The system's diagnostic modules allow setup, instrument alignment, and motor operations. The customized tools, labware, and liquid and particle transfer patterns allow different applications to be programmed and performed. The database allows method and parameter storage. Robotic and computer interfaces allow communication between instruments.

Persons having skill in the art will recognize the various robotic platforms capable of carrying out the HTP engineering methods of the present disclosure. Table 1 below provides a non-exclusive list of scientific equipment capable of carrying out each step of the HTP engineering steps of the present disclosure, such as those described in FIGS. 36A-36B.

TABLE 1
Non-exclusive list of Scientific Equipment Compatible
with the HTP engineering methods of the disclosure

| | Equipment | | Compatible Equipment |
|---|---|---|---|
| | Type | Operation(s) performed | Make/Model/Configuration |
| Acquire and build | liquid handlers | Hitpicking (combining by | Hamilton Microlab STAR, |
| DNA pieces | | transferring) | Labcyte Echo 550, Tecan EVO |
| | | primers/templates for PCR | 200, Beckman Coulter Biomek |
| | | amplification of DNA | FX, or equivalents |
| | | parts | |
| | Thermal cyclers | PCR amplification of | Inheco Cycler, ABI 2720, ABI |
| | | DNA parts | Proflex 384, ABI Veriti, or |
| | | | equivalents |
| QC DNA parts | Fragment | gel electrophoresis to | Agilent Bioanalyzer, AATI |
| | analyzers | confirm PCR products of | Fragment Analyzer, or |
| | (capillary | appropriate size | equivalents |
| | electrophoresis) | | |
| | Sequencer | Verifying sequence of | Beckman Ceq-8000, Beckman |
| | (sanger: | parts/templates | GenomeLab ™, or equivalents |
| | Beckman) | | |
| | NGS (next | Verifying sequence of | Illumina MiSeq series |
| | generation | parts/templates | sequences, illumina Hi-Seq, Ion |
| | sequencing) | assessing concentration of | torrent, pac bio or other |
| | instrument | DNA samples | equivalents |
| | nanodrop/plate | | Molecular Devices SpectraMax |
| | reader | | M5, Tecan Ml000, or |
| | | | equivalents. |
| Generate DNA | liquid handlers | Hitpicking (combining by | Hamilton Microlab STAR, |
| assembly | | transferring) DNA parts | Labcyte Echo 550, Tecan EVO |
| | | for assembly along with | 200, Beckman Coulter Biomek |
| | | cloning vector, addition of | FX, or equivalents |
| | | reagents for assembly | |
| | | reaction/process | |
| QC DNA | Colony pickers | for inoculating colonies in | Scirobotics Pickolo, Molecular |
| assembly | | liquid media | Devices QPix 420 |
| | liquid handlers | Hitpicking | Hamilton Microlab STAR, |
| | | primers/templates, diluting | Labcyte Echo 550, Tecan EVO |
| | | samples | 200, Beckman Coulter Biomek |
| | | | FX, or equivalents |
| | Fragment | gel electrophoresis to | Agilent Bioanalyzer, AATI |
| | analyzers | confirm assembled | Fragment Analyzer |
| | (capillary | products of appropriate | |
| | electrophoresis) | size | |
| | Sequencer | Verifying sequence of | ABI3730 Thermo Fisher, |
| | (sanger: | assembled plasmids | Beckman Ceq-8000, Beckman |
| | Beckman) | | GenomeLab ™, or equivalents |
| | NGS (next | Verifying sequence of | Illumina MiSeq series |
| | generation | assembled plasmids | sequences, illumina Hi-Seq, Ion |
| | sequencing) | | torrent, pac bio or other |
| | instrument | | equivalents |
| Prepare base strain | centrifuge | spinning/pelleting cells | Beckman Avanti floor |
| and DNA assembly | | | centrifuge, |
| | | | Hettich Centrifuge |
| Transform DNA | Electroporators | electroporative | BTX Gemini X2, BIO-RAD |
| into base strain | | transformation of cells | MicroPulser Electroporator |
| | Ballistic | ballistic transformation of | BIO-RAD PDS1000 |
| | transformation | cells | |
| | Incubators, | for chemical | Inheco Cycler, ABI 2720, ABI |
| | thermal cyclers | transformation/heat shock | Proflex 384, ABI Veriti, or |
| | | | equivalents |
| | Liquid handlers | for combining DNA, cells, | Hamilton Microlab STAR, |
| | | buffer | Labcyte Echo 550, Tecan EVO |
| | | | 200, Beckman Coulter Biomek |
| | | | FX, or equivalents |
| Integrate DNA into | Colony pickers | for inoculating colonies in | Scirobotics Pickolo, Molecular |
| genome of base strain | | liquid media | Devices QPix 420 |
| | Liquid handlers | For transferring cells onto | Hamilton Microlab STAR, |
| | | Agar, transferring from | Labcyte Echo 550, Tecan EVO |
| | | culture plates to different | 200, Beckman Coulter Biomek |
| | | culture plates (inoculation | FX, or equivalents |
| | | into other selective media) | |
| | Platform | incubation with shaking of | Kuhner Shaker ISF4-X, Infors- |
| | shaker- | microtiter plate cultures | ht Multitron Pro |
| | incubators | | |
| QC transformed | Colony pickers | for inoculating colonies in | Scirobotics Pickolo, Molecular |
| strain | | liquid media | Devices QPix 420 |
| | liquid handlers | Hitpicking | Hamilton Microlab STAR, |
| | | primers/templates, diluting | Labcyte Echo 550, Tecan EVO |
| | | samples | 200, Beckman Coulter Biomek |
| | | | FX, or equivalents |
| | Thermal cyclers | cPCR verification of | Inheco Cycler, ABI 2720, ABI |
| | | strains | Proflex 384, ABI Veriti, or |
| | | | equivalents |
| | Fragment | gel electrophoresis to | Infors-ht Multitron Pro, Kuhner |
| | analyzers | confirm cPCR products of | Shaker ISF4-X |
| | (capillary | appropriate size | |
| | electrophoresis) | | |
| | Sequencer | Sequence verification of | Beckman Ceq-8000, Beckman |
| | (sanger: | introduced modification | GenomeLab ™, or equivalents |
| | Beckman) | | |
| | NGS (next | Sequence verification of | Illumina MiSeq series |
| | generation | introduced modification | sequences, illumina Hi-Seq, Ion |
| | sequencing) | | torrent, pac bio or other |
| | instrument | | equivalents |
| Select and | Liquid handlers | For transferring from | Hamilton Microlab STAR, |
| consolidate | | culture plates to different | Labcyte Echo 550, Tecan EVO |
| QC'd strains | | culture plates (inoculation | 200, Beckman Coulter Biomek |
| into test plate | | into production media) | FX, or equivalents |
| | Colony pickers | for inoculating colonies in | Scirobotics Pickolo, Molecular |
| | | liquid media | Devices QPix 420 |
| | Platform | incubation with shaking of | Kuhner Shaker ISF4-X, Infors- |
| | shaker- | microtiter plate cultures | ht Multitron Pro |
| | incubators | | |
| Culture strains | Liquid handlers | For transferring from | Hamilton Microlab STAR, |
| in seed plates | | culture plates to different | Labcyte Echo 550, Tecan EVO |
| | | culture plates (inoculation | 200, Beckman Coulter Biomek |
| | | into production media) | FX, or equivalents |
| | Platform | incubation with shaking of | Kuhner Shaker ISF4-X, Infors- |
| | shaker- | microtiter plate cultures | ht Multitron Pro |
| | incubators | | |
| | liquid | Dispense liquid culture | Well mate (Thermo), |
| | dispensers | media into microtiter | Benchcel2R (velocity 11), |
| | | plates | plateloc (velocity 11) |
| | microplate | apply barcoders to plates | Microplate labeler (a2+ cab - |
| | labeler | | agilent), benchcell 6R |
| | | | (velocity 11) |
| Generate product | Liquid handlers | For transferring from | Hamilton Microlab STAR, |
| from strain | | culture plates to different | Labcyte Echo 550, Tecan EVO |
| | | culture plates (inoculation | 200, Beckman Coulter Biomek |
| | | into production media) | FX, or equivalents |
| | Platform | incubation with shaking of | Kuhner Shaker ISF4-X, Infors- |
| | shaker- | microtiter plate cultures | ht Multitron Pro |
| | incubators | | |
| | liquid | Dispense liquid culture | well mate (Thermo), |
| | dispensers | media into multiple | Benchcel2R (velocity 11), |
| | | microtiter plates and seal | plateloc (velocity 11) |
| | | plates | |
| | microplate | Apply barcodes to plates | microplate labeler (a2+ cab - |
| | labeler | | agilent), benchcell 6R |
| | | | (velocity 11) |
| Evaluate | Liquid handlers | For processing culture | Hamilton Microlab STAR, |
| performance | | broth for downstream | Labcyte Echo 550, Tecan EVO |
| | | analytical | 200, Beckman Coulter Biomek |
| | | | FX, or equivalents |
| | UHPLC, HPLC | quantitative analysis of | Agilent 1290 Series UHPLC |
| | | precursor and target | and 1200 Series HPLC with |
| | | compounds | UV and RI detectors, or |
| | | | equivalent; also any LC/MS |
| | LC/MS | highly specific analysis of | Agilent 6490 QQQ and 6550 |
| | | precursor and target | QTOF coupled to 1290 Series |
| | | compounds as well as side | UHPLC |
| | | and degradation products | |
| | Spectrophotometer | Quantification of different | Tecan M1000, spectramax M5, |
| | | compounds using | Genesys 10S |
| | | spectrophotometer based | |
| | | assays | |
| Culture strains | Fermenters: | incubation with shaking | Sartorius, DASGIPs |
| in flasks | | | (Eppendorf), BIO-FLOs |
| | | | (Sartorius-stedim). Applikon |
| | Platform | | innova 4900, or any equivalent |
| | shakers | | |
| Generate product | Fermenters: DASGIPs (Eppendorf), BIO-FLOs (Sartorius-stedim) | | |
| from strain | | | |
| Evaluate | Liquid handlers | For transferring from | Hamilton Microlab STAR, |
| performance | | culture plates to different | Labcyte Echo 550, Tecan EVO |
| | | culture plates (inoculation | 200, Beckman Coulter Biomek |
| | | into production media) | FX, or equivalents |
| | UHPLC, HPLC | quantitative analysis of | Agilent 1290 Series UHPLC |
| | | precursor and target | and 1200 Series HPLC with |
| | | compounds | UV and RI detectors, or |
| | | | equivalent; also any LC/MS |
| | LC/MS | highly specific analysis of | Agilent 6490 QQQ and 6550 |
| | | precursor and target | QTOF coupled to 1290 Series |
| | | compounds as well as side | UHPLC |
| | | and degradation products | |
| | Flow cytometer | Characterize strain | BD Accuri, Millipore Guava |
| | | performance (measure | |
| | | viability) | |
| | Spectrophotometer | Characterize strain | Tecan M1000, Spectramax M5, |
| | | performance (measure | or other equivalents |
| | | biomass) | |

Transfer Function

The Transfer Function application, International Application No. PCT/US18/60120, provides a robust method for reliably predicting the values of key performance indicators (e.g., yield, productivity, titer) of microbes in larger-scale, low-throughput conditions based on smaller-scale, high-throughput microbe performance. This is especially useful for metabolic optimization of organisms for mass-production of chemical targets. Embodiments may employ an optimized statistical model for the prediction.

According to embodiments of the disclosure, a transfer function is a statistical model for predicting performance in one context based on performance in another, where the primary goal is to predict the performance of samples at a larger-scale from their performance at a smaller-scale. In embodiments, the transfer function involves simple, one-factor linear regression between small-scale values and large-scale values, along with optimizations discovered by the inventors. In other embodiments, the transfer function may employ multiple regression.

To build these regression models, embodiments of the disclosure use an input model to summarize the performance of a strain in the high-throughput context (e.g., a statistical plate model), and then use a separate model (e.g., a transfer function) to predict the performance of a strain across multiple runs in the lower-throughput context. The plate model may, for example, be used to model the performance (e.g., yield, productivity, viability) of multiple replicates of the same strain in a 96-well plate. According to embodiments of the disclosure, a programmed computer, which may, for example, be the prediction engine or the computing portion of the analysis equipment, generates the input model, generates the transfer function, applies the transfer function to the input model output to predict performance, or performs any combination thereof.

The following optimization considerations may be taken into account both in the transfer function and in the statistical plate summarization models, and in building more complicated, nonlinear machine-learning models for predicting performance in a lower throughput context from performance in a higher throughput context:
accounting for bias due to both the plate and the location on the plate (e.g., row-column location, edge location),
plate characteristics, such as media type/lot, shaker location bias,
process characteristics, like the number of times the glycerol stock used to inoculate wells has been used, and which type of machines (e.g., incubators, fermenters, measurement equipment) were used at both the lower and higher-throughput steps,
sample characteristics (such as cell lineage or presence/absence of known genetic markers)

Approaches for building a robust and reliable transfer function for accurately predicting key performance indicators at larger scale based on smaller-scale high-throughput measurements are presented below.

This disclosure first presents a basic linear model according to embodiments of the disclosure. The disclosure then presents optimizations implemented algorithmically according to embodiments of the disclosure. According to embodiments, the transfer function development tool includes an infrastructure to implement further optimizations after the data is in an ingestible format. The following examples are based on the problem of predicting bioreactor (larger-scale, lower-throughput) productivities (g/L/h) and yields (wt %) of an amino acid based on titers of the amino acid at 24 and 96 hours, respectively, in 96-well plates (smaller-scale, higher-throughput) for individual strains.

The Basic Transfer Function: Plate-Tank Correlation Function

The most basic form of the transfer function is a single-factor linear regression of the form y=mx+b, where x is the value obtained in small-scale, high-throughput screening, y is the value obtained in large-scale, low-throughput screening, and m and b are the slope and y intercept, respectively, of the fit line. Embodiments may also employ multiple regression to predict dependent variable y based on multiple independent variables xᵢ. The correlation between x and y values at the two scales can be used as a measure of how effective this basic approach is; thus it may be called the “plate-tank correlation.”

Even this basic form of the transfer function incorporates an inventive optimization. Instead of simply using the mean performance of a strain to obtain a single value for the strain from the high-throughput screening to correlate to the lower-throughput values, embodiments of the disclosure employ a linear model that corrects for plate location bias, among other factors. Other embodiments employ non-linear models, and account for other aspects of the plate model.

The plate-tank correlation (i.e., transfer) function not only predicts performance of samples that have not been tested at a lower-throughput, larger scale. It also may be used to assess the effectiveness of the physical plate model. The physical plate model is a collection of media and process constraints designed to make the values obtained at small-scale in high-throughput as predictive as possible of the values obtained at large scale. The correlation coefficient of the plate-tank correlation function indicates, among other things, how well the plate model is fulfilling its purpose. The plate model may incorporate, but is not limited to, physical features (which may function as independent variables in the plate model) such as:
media formulation and preparation (e.g. media lots)
diluent type
inoculation volume
labware
shaking time, temperature and humidity

In embodiments of the disclosure, the plate-tank correlation function is used to optimize the physical plate model. In embodiments the physical plate model mimics the microbial fermentation process at tank scale—to physically model tank performance via implementation in the plates.

Plate Model

The performance of a strain in the high-throughput context (e.g., in a small-scale, plate environment) may be determined via a Least Squares Means (LS-Means) method, according to embodiments of the disclosure. LS-Means is a two-step process by which first a linear regression is fit, and then that fit model predicts the performance over the Cartesian set of all categorical features, and the mean of all numerical features. The features of the model relate the physical plate model to a statistical plate model, and describe conditions under which that experiment was conducted, and include the optimizations listed above (e.g., location on the plate, plate characteristics, process characteristics, sample characteristics).

The model form of the first step is:

titerᵢ=β_{s[i]}+Σ_{f}β_{f}x_{f}[i]

There is an inferred additive coefficient, βₛ, for the strain's effect (titer in this example), and then each additional feature used in the model. The first term βₛ is the effect (here, titer) of the strain replicate indexed by i. Then each additional term β_{f} is the weighting assigned to feature, f, (e.g., plate location) and x_{f[i]} is the value of the feature for the strain replicate indexed by i.

As an example, one such model might be:

titerᵢ=β_{s[i]}+βₚₗₐₜₑ plateᵢ

In this model, the feature is the particular plate on which the strain is grown. This model includes a coefficient βₚₗₐₜₑ for each strain and each plate indexed by i in the particular experiment. The model may be fit using ridge regression with a penalty to improve numerical stability.

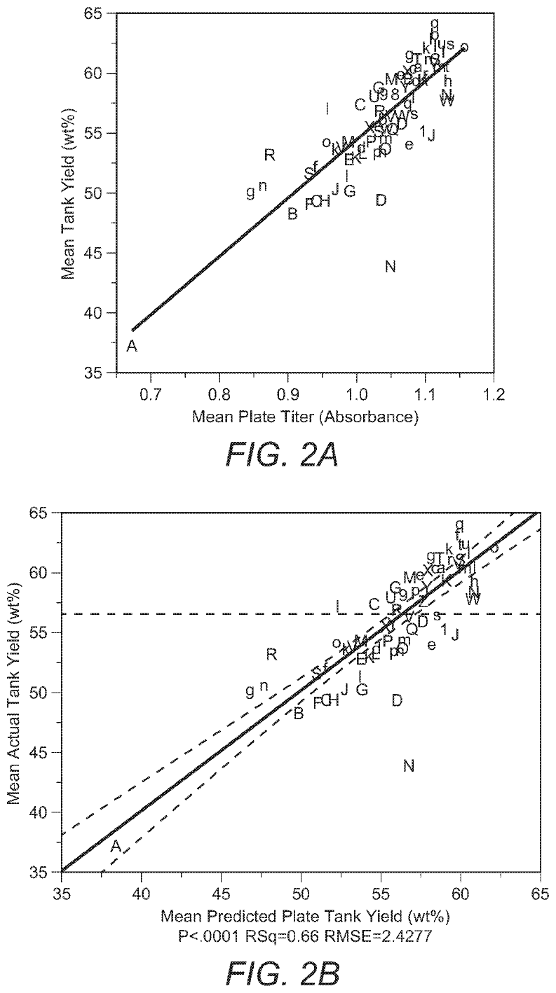

The second step again takes all possible combinations of the factors (e.g., particular plate and location on the plate for all strains) and makes predictions on those synthetic values using the statistical plate model equation to simulate what would occur in the event a strain was run in each scenario, and finally the mean performance of scenarios by strain is taken. This is the final point estimate associated with the plate performance (e.g. the x-axis plate performance value in FIG. 2A), and that is correlated with a summary of tank performance (e.g. the y-axis tank performance value in FIG. 2A).

FIG. 2A illustrates an example of a correlation according to embodiments of the disclosure. FIG. 2A illustrates a comparison of measured bioreactor (tank, larger scale) vs. plate (smaller scale) values for individual strains. The dataset includes high-throughput measurements (using the plate model to determine yield), and associated bioreactor measurements (e.g., yield) for producing an amino acid. Average plate titers (incorporating estimated plate bias) per strain are on the x-axis, and average bioreactor (e.g., tank, fermenter) yields (wt %) per strain are on the y-axis. Each point (letter) corresponds to a single strain.

For purposes of prediction, such plots may be examined in terms of how well the model's predicted performance matches up with the actual performance, which for the simple case shown in the figure is the regression plot with a rescaled x-axis. FIG. 2B illustrates a comparison of actual yield values to simple linear predicted yield values for a bioreactor (tank). The dotted horizontal line is the global mean of actual tank values, and the dotted diagonal lines represent a 95% confidence interval of the actual location of the fit line. Predicted P, RSq, and RMSE are the primary metrics of model performance here, with Predicted P being the P-value of the fit, RSq being the R² of the correlation, and RMSE being the root mean squared error of the predictions. Of these, RMSE is the most useful for optimization purposes, since it is the most direct measure of prediction accuracy.

Optimizations

Outliers

In examining the plots above, some strains behave very differently from the rest and are spatially isolated. These outliers can be classified into two types: Type 1 outliers that represent extreme values in performance, y axis, e.g., yield, and Type 2 outliers that represent, otherwise referred to as “high leverage points” that represent extreme values in the x axis. Type 1 outliers are those strains that are far away from the fit line; i.e., they are predicted poorly (the strain labeled N in the lower right quadrant of FIG. 2B is an example). Such strains affect the fit of the model and can impair predictivity for all other strains while still being poorly predicted themselves. One optimization is to remove such strains to improve the overall predictive power of the model. Another optimization is to add factors to the transfer function model, or to the model that summarizes the strain performance at the higher-throughput level (e.g., plate model incorporating plate location bias, or genetic factors).

Type 2 outliers are those that are on or close to the fit line but still distant from other strains (the strain labeled A in the lower left corner is an example in FIG. 2B). Distance can be measured in a number of ways including: distance from the centroid of the other strains, or distance to the nearest other strain. Type 2 outliers exert outsize influence on the simple linear model. The purpose of the model is to predict, as accurately as possible, the performance of the remaining strains. Thus, embodiments of the disclosure optimize with regard to Type 2 outliers by removing them (in conformance with general statistical practice), or alternatively, by optimizing the model by adding predictive factors.

In the case of optimizing by removal of the outlier, embodiments of the disclosure provide at least two approaches to labeling a strain as an outlier to be removed:

The first is on the basis of the strain appearing repeatedly as an outlier and on having a meaningful rationale based on the unusual characteristics of the strain or its performance at a larger scale to exclude it as not representative of the bulk of strains. For instance, the A strain in FIG. 2B is a progenitor of the other strains in the model, but genetically and in performance at scale rather distant from them. The N strain has a modification known to give good results in the plate but to fails to consume enough glucose at larger scales.

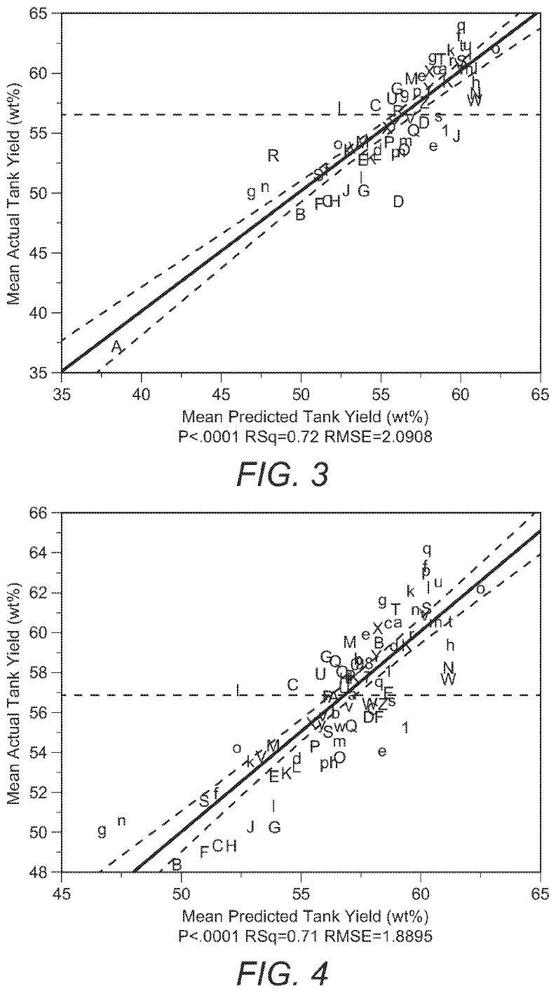

The second outlier-labeling method is to assign a “leverage metric” to each strain and consider it an outlier if the change in the metric due to removal of the strain exceeds a pre-defined cutoff (“leverage threshold”). For instance, the leverage metric may represent the percentage difference in RMSE with and without the strain in the model, and the cutoff may be a 10% improvement. In this case, the results of removing the N strain are depicted in FIG. 3.

FIG. 3 is a plot equivalent to that of FIG. 2B, except with Type 1 outlier strain N removed. Removing the N strain decreases the RMSE from 2.43 to 2.09, or 14%, which is higher than the currently used cutoff of 10%. Thus, the prediction engine would identify the outlier for removal.

Care should be taken in removing outlier strains (e.g., setting the outlier cutoff too low) because of the danger of overfitting, i.e., building a model that predicts a small subset of strains very well but does poorly when used on the broader population. One way to protect against this is to use a cut-off that is weighted by the number or fraction of candidate strains in the model. For instance, if the base cutoff is 10% and there are 100 strains that could be included the model, the cutoff for removing the first strain may be 0.1/0.99, the cutoff for removing the second strain could be 0.1/0.98, the cutoff for the third 0.1/0.97, etc.

After removing one Type 2 outlier and four Type 1 outliers, the fit of FIG. 3 becomes as shown in FIG. 4. FIG. 4 is a plot equivalent to that of FIG. 2B, except with four Type 1 outliers and one Type 2 outlier removed. Note that RSq and RMSE are both improved in FIG. 4, by approximately 6% and 21%, respectively, relative to the model in FIG. 2B.

Genetic and Other Factors

Genetic or other characteristics of the samples (including process aspects, such as the lot number of the media used for growing the strains) can also be useful for improving predictive power as factors in the transfer function, especially given that a high-throughput plate model alone is unlikely to completely recapitulate the conditions that samples will be subjected to at a larger scale. In the case of metabolic engineering, in particular, it is impossible to reproduce conditions in a five-liter or larger bioreactor, such as the effects of fluid dynamics, shear stresses, and diffusion of oxygen and nutrients, in 200-μL wells in a plate. Work towards improving the physical plate model based on factors such as media composition, method of media preparation, compounds measured, and timing of measurements has downsides in being time-consuming and expensive, and possibly making it difficult to compare samples run under a new plate model to those run under the old. Thus, embodiments of the disclosure identify and make use of other predictive factors of the plate model to improve predictions. Some of those other factors, according to embodiments of the disclosure, include:
accounting for bias due to location of strain on a plate
plate characteristics, like media type/lot, shaker location bias
process characteristics, such as the number of times the glycerol stock used to inoculate wells has been used and which type of machines were used at both the lower and higher-throughput steps
sample characteristics (such as cell lineage or presence/absence of known genetic markers)

The inventors have found genetic factors, in particular, to be useful in improving the transfer function for metabolically engineered strains—for example, incorporating information about changes that lead to differences in gene regulation.

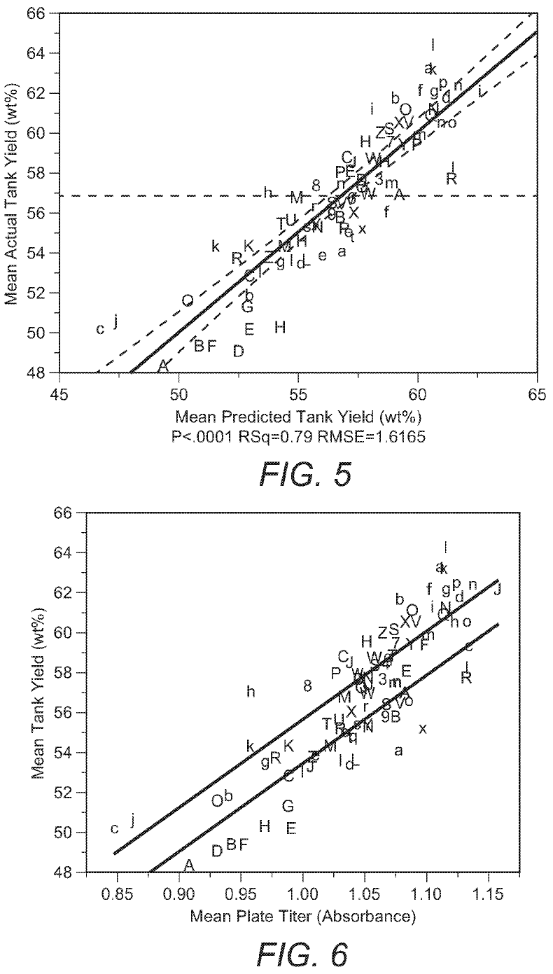

FIG. 5 depicts the result of applying a correction to all the strains in FIG. 4 based on whether or not they have a certain genetic modification (e.g., a start-codon swap in a particular gene). As an example, for a multiple regression transfer function model, the adjustment/correction accounting for the presence or absence of the start-codon swap may take the form of adding a performance component mᵢxᵢ or a performance component mⱼxⱼ, respectively, to the mean tank yield performance of the strains predicted by the transfer function. (Note that the weight m may take on negative values.) In embodiments, mᵢ may take on a single value, and x is +1 or −1 depending upon whether the modification is present or not, respectively. In other embodiments, mᵢ may take on a single value, and x is +1 or 0.

FIG. 5 is equivalent to FIG. 4, except it includes a correction factor for the presence or absence of a start codon swap in the aceE gene. This correction increases the RSq (R squared) from 0.71 to 0.79 and decreases the RMSE from 1.9 to 1.6 (16%).

FIG. 6 is a regression plot of the model shown in FIG. 5. The regression plot (FIG. 6) shows that essentially two regression lines are used, depending on whether the modification is present (upper line) or absent (lower line).

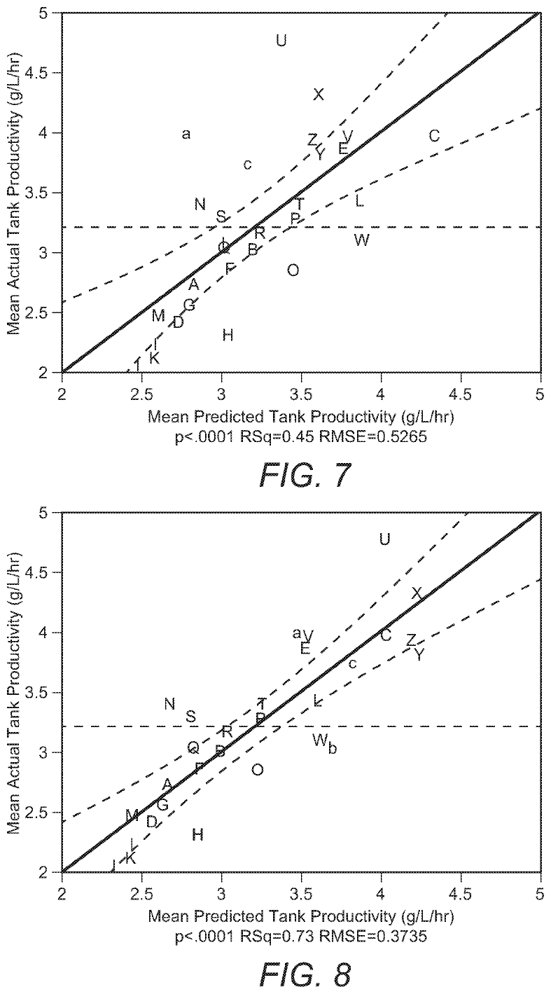

FIG. 7 illustrates a productivity model without correction for genetic factors. The results of correcting for genetics are even more striking in the productivity model. Without correcting for a genetic change that the plate model fails to recapitulate (e.g., a promoter swap), the model is as shown in FIG. 7.

Including the correction for the presence or absence of this modification yields the model shown in FIG. 8. FIG. 8 illustrates the productivity model of FIG. 7 after correction for a genetic factor (e.g., a particular promoter swap). A promoter swap is a promoter modification, including insertion, deletion, or replacement of a promoter.

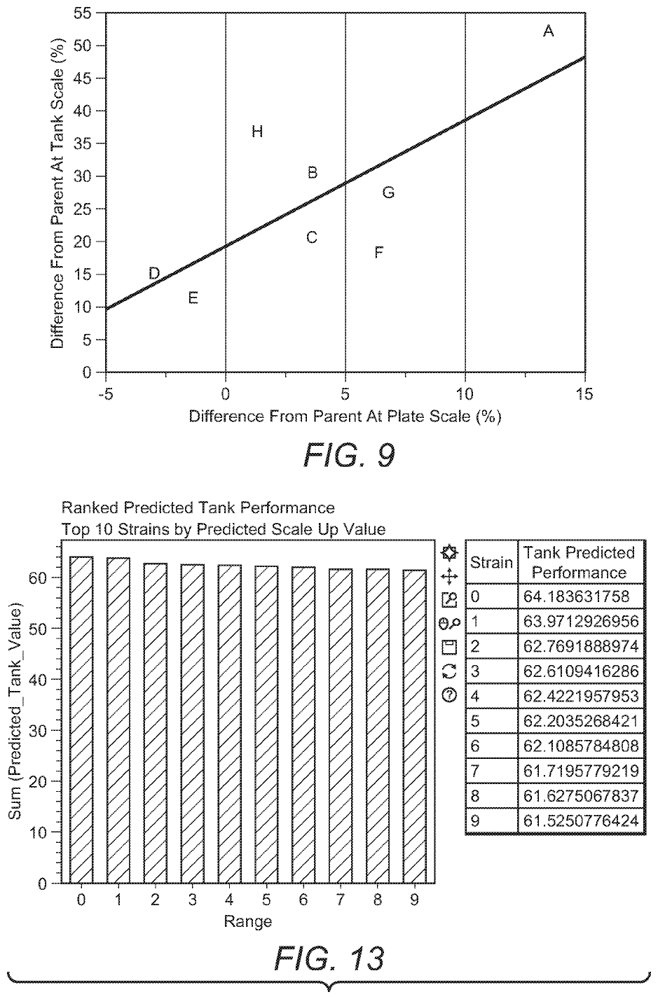

Including this factor in the model (e.g., multiple regression model) increases RSq from 0.45 to 0.73 and reduces RMSE from 0.53 to 0.37 (30%), which is an impactful increase in predictive power. In fact, examining the improvement in plate performance (“hts_prod_difference”) versus the improvement in bioreactor (tank) performance (tank_prod_difference) for strains harboring this modification (with two outliers removed) and fitting them to a line yields FIG. 9.

FIG. 9 illustrates improvement in the high-throughput productivity-model performance (x-axis) versus improvement in actual productivity in low-throughput bioreactors (e.g., tanks) (y-axis) for strains harboring the same promoter swap as in FIG. 8.

The equation of the fit line is 19+1.9*hts_prod_difference, meaning that a strain harboring this change that is indistinguishable from its parent in the plate model can be expected to perform approximately 20% better than its parent at scale, a major improvement that the plate model alone cannot accurately predict. Even strains that the plate model alone predicts will be worse at the plate level than parent (like D and E in the plot of FIG. 9) are in fact much better than parent at tank scale. Including a factor for this change in the model accurately predicts these effects in new strains and avoids losing such strains as false negatives.

Groups of genetic factors may also be useful in prediction, as a result of epistatic interactions, in which the effect of two or more modifications in combinations differs from what would be expected from the additive effects of the modifications in isolation. For a more detailed explanation of epistatic effects, please refer to PCT Application No. PCT/US16/65465, filed Dec. 7, 2016, incorporated by reference in its entirety herein.

Another factor is lineage. Lineage is similar to genetic factors in that it is hereditary, but lineage takes into account both the known and unknown genetic changes that are present in a strain compared to other strains in other lineages. Embodiments of the disclosure employ lineage as a factor to build a directed acyclic graph of strain ancestry, and test the most connected nodes (i.e., the progenitor strains that have been used most frequently as targets for further genetic modifications or have the largest number of descendants) for their utility as predictive factors.

Modifications to Transfer Function Output

The simplest way to use transfer function output is to use the output as a prediction of performance at scale. Another approach is to apply the percent change in transfer predictions between parent and daughter strain to the actual large-scale performance of the parent (i.e., prediction=parent_performance_at_scale+parent_performance_at_scale*(TF_output(daughter)−TF_output(parent))/TF_output(parent)), where parent_performance_at_scale is the observed performance of the parent strain at scale (i.e., larger scale), TF_output(strain) is the predicted performance of a strain “strain” due to application of the transfer function, and the daughter strain is a version of the parent strain as modified by one or more genetic modifications. This has the benefit of removing noise associated with the influence of the parent on the daughter's performance at scale, but assumes that such influence exists; i.e., it assumes that the transfer function's error in predicting the daughter's performance will be of approximately the same magnitude and sign as the error in predicting the parent.

Other Statistical Models

The above assumes the transfer function uses simple linear and multiple regression models, but more sophisticated linear models, such as ridge regression or lasso regression, may also be employed in embodiments of the disclosure. Additionally, non-linear models, including polynomial (e.g., quadratic) or logistic fits, or nonlinear machine learning models such a K-nearest neighbors or random forests may be employed in embodiments. More sophisticated cross-validation approaches may be used to avoid overfitting.

### Algorithm Example

In embodiments, the decisions for what samples (strains) to include or exclude as outliers and what potential factors to include to improve predictive power are implemented in an algorithm to ensure reproducibility, explore as many possibilities for improvement as possible, and reduce the influence of subconscious bias. A variety of approaches may be adopted, and an example of one such cyclic/iterative process is presented below, in which the small scale, high throughput environment may correspond to a plate environment, and the large scale, low throughput environment may correspond to a tank environment.
1. Start with a set of strains, using performance measurement(s) (e.g., amino acid titer) as sole factor(s) for developing the predictive model (e.g., linear regression)
   a. These are strains for which actual plate and tank performance data are known.
2. Identify the strain whose removal from the transfer function model most improves RMSE for the model (“the Outlier”).
   a. Alternatively, identify for potential removal from the model the strain that has the greatest prediction error (predicted vs. measured performance for the strain).
3. If the RMSE improvement from removing the strain is greater than a predefined cut-off, proceed to Step 4; otherwise go to Step 10.
4. Identify potential predictive factors that apply to the Outlier that are not present in all other strains currently included in the model (because factors that are equivalent in all strains are not useful for overall predictive power), and are not already included as factors in the model. Optionally, the algorithm may identify factors present in at least one other strain, while still meeting the above conditions.
   a. Factors that are characteristic of the Outlier strain may include, for example, genetic changes known to have been made, lineage (history of strain ancestry), phenotypic characteristics, growth rate.
   b. Note that if a factor is in only one strain, the algorithm may adjust the model to correct for that single strain, but usually modifying the model to account for a single strain may not be an expected objective. Also, if the factor is in all other strains, then it has no predictive value.
   c. Note that embodiments may employ a machine learning model that would automatically perform this function, but that identifying the factors for the model may reduce the resource burden on the machine learning model.
5. If the list from Step 4 is empty, exclude the Outlier from the model and go to Step 2.
6. Otherwise, provisionally apply the factors from Step 4 in the model.
   a. As noted above, embodiments may employ a simple linear regression transfer function such as y=m₁x₁+b, where x₁ is the performance of a strain on the plate, and m₁ is a weight (slope) applied to x₁. In embodiments, the model may be refined by adding weighted factors (regression coefficients) to generate a multiple regression model of the form y=m₁x₁+m₂x₂+ . . . +m_{N}x_{N}+b, where x₁ is the performance of a strain on the plate, the other xᵢ (i≠1) represent factors other than performance x₁, m₁ is a weight applied to x₁, and mᵢ is a weight applied to factor xᵢ. In embodiments, x₁ may represent the output of a plate model. In embodiments, all xᵢ may represent the output of a plate model.
   b. In embodiments, the factors may be added one at a time, and the weighting adjusted, until error (or P value) is reduced by a satisfactory amount before adding the next factor.
7. The algorithm may remove factors (e.g., x values in the multiple regression equation) if the factors do not improve the error of the model by an error threshold or if they have a P-value above a P-value threshold. For example, embodiments of the disclosure may remove particular genetic factors (i.e., genetic modifications known to have been made in the strain) from the regression model (prediction function) if those factors do not improve the error by an error threshold or if they have a P-value above a P-value threshold.
8. According to embodiments of the disclosure, if any remaining genetic factors are part of a group having a high variance inflation factor (e.g., >3, indicative of colinearity between factors), the prediction engine may keep only the genetic factor with the lowest P-value within each group. A high variance inflation indicates a high correlation between factors. Including highly correlated factors would not provide much predictive value and could cause overfitting. According to embodiments of the disclosure, the prediction engine may use variance inflation factor to measure the correlation between factors, and start with removing highly correlated factors until a satisfactory a satisfactory variance inflation factor is reached.
9. If all the genetic changes from Step 4 have been removed at this point, remove the Outlier strain from the model, and return to Step 2.
   a. If the condition is true, the algorithm has determined that the algorithm cannot be satisfactorily improved without removing the Outlier.
10. After iterating through Steps 2-9 or jumping here from Step 3, remove any factors that apply to none or all of the remaining strains. Optionally, remove any genetic factors that only apply to one strain.

The result of the above algorithm may be an improved model with some outliers removed and the model adjusted to account for more factors. The outputs include strains used to develop the model and factors used in the model, along with their weights.

According to embodiments of the disclosure, the prediction engine may compare performance error metrics for a plurality of prediction functions, and rank the prediction functions based at least upon the comparison. Referring to the algorithm above, the prediction engine may compare the predictive performance of models created by different iterations (e.g., different outliers removed, different factors added). According to embodiments, the prediction engine may compare the predictive performance of models created by different techniques, e.g., ridge regression, multiple regression, random forest.

Embodiments of the disclosure test new versions of the transfer function and monitor its performance by measuring actual performance of the strain at large scale. A new transfer function's predictions may be back-tested against other versions of the transfer function and compared in performance on historical data. Then the transfer function may be forward-tested in parallel with other versions on new data. Metrics of performance (such as RMSE) may be monitored over time, so that improvements may be made quickly if performance begins to fall off. (Similar processes can be used to improve and monitor the plate model, and the two processes can also be combined to include a decision point as to whether efforts toward improvement should focus on the transfer function or the plate model.)

In embodiments, if the transfer function fails to accurately predict strain performance at the bioreactor scale, physical adjustments may be made to the physical plate cultivation model. As with adjustments to the parameters/weights of the mathematical model, physical changes to the physical plate model may be made based on the phenotype of interest. Several changes may be made and evaluated to determine which physical plate model(s) yield the best transfer function. Examples of changes include, but are not limited to, media composition, cultivation time, compounds measured, and inoculation volume.

### EXPERIMENTAL EXAMPLES

The following two examples show use of embodiments of the disclosure to produce different products of interest in different organisms.

### Example 1

When fitting a statistical model for predicting performance of microbes at a larger scale (e.g., tank) based on a smaller scale (e.g., plate), embodiments of the disclosure use multiple metrics as well as standard statistical techniques for fitting the model. In these experiments, the prediction engine uses multiple plate measurements per plate to derive a predictive function, and the plate values are based on statistical plate models that are themselves based on raw, measured physical plate data. This Example 1 covers one main product, a polyketide produced by a Saccharopolyspora bacterium.

In the following discussion, embodiments of the disclosure make use of the standard adjusted R², root mean squared error (RMSE) for a set of test strains, and a leave one out cross validation (“LOOCV”) metric.

RMSE: A set of strains, the training strains (marked as “train”), were used to fit the model. Then the prediction engine screened many new strains in plates (not the strains used to train the model), and promoted a subset of those strains to tanks (i.e., selected those strains with good statistics to be generated in tanks at the larger scale). The prediction engine computed

$$RMSE = \sqrt{\sum \frac{{( {tank}_{actual} - {tank}_{predicted} )}^{2}}{n}}$$

for this set of test strains, where n is the number of test strains, and the variable tank is the performance metric of interest (e.g., yield, productivity) at tank scale.

LOOCV: According to embodiments of the disclosure, for any new model, according to LOOCV the prediction engine iterated through the set of training strains. At each step, the prediction engine removed a strain from the training data, fitted the model using the remaining training data, and computed the RMSE for the removed, former training strain as a test strain (see previous discussion of RMSE). The prediction engine set RMSEᵢ to be the RMSE with the i^{th} strain removed. The prediction engine then computed the mean of this set of RMSE values so

$$LOOCV = \frac{{\sum }_{i} {RMSE}_{i}}{m}$$

where m is the total number of strains in the training set.

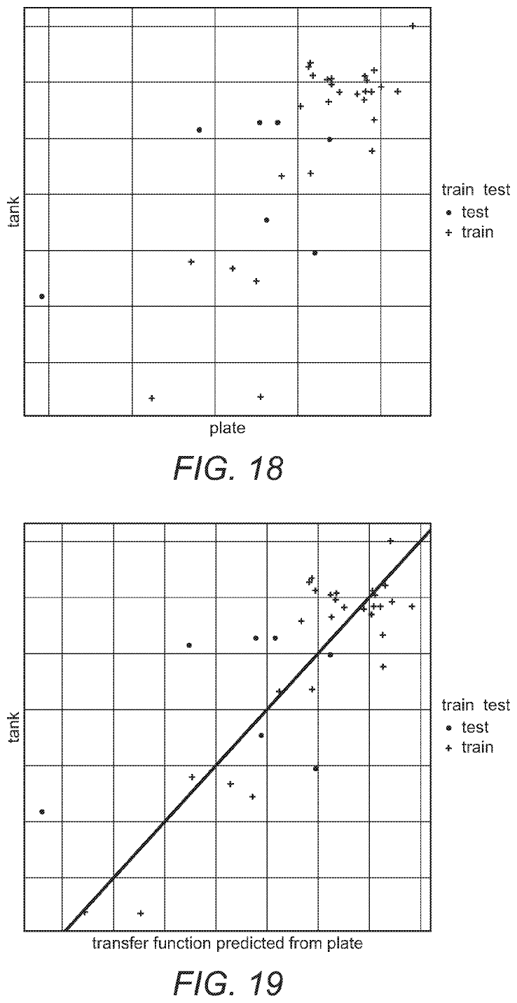

FIG. 18 is a graph of the plate vs. tank values for the primary metric of interest. The figure shows a reasonable linear relationship. If the prediction engine fits the simple linear model tank=b+m₁*plate_value₁ on the microbes marked as train, where b=−3.0137, m₁=0.0096 and plate_valueᵢ is a polyketide value in mg/L processed by the statistical plate model, then the adjusted R\hat{ }2 is 0.65, the leave one out CV is 2.65, and the RMSE of the test set is 5.2152.

If the prediction engine instead fits the linear-regression model tank=b+m₁*plate_value₁+m₂*plate_value₁*plate_value₂, where b=0.7728, m₁=0.0325, m₂=0.0000646, and both plate_values are for two different polyketides (in mg/L) processed by the statistical plate model, the prediction engine provides a much more predictive transfer function, as shown in the FIG. 19. Note that the plate values plate_value₁, plate_value₂, etc. represent assays on the same plate, and can be the same or different assays on the plate, e.g., all product of interest assays (e.g., yield), or instead product of interest and another assay, such as biomass or glucose consumption. According to embodiments of the disclosure, the plate value or tank value may represent a mean amount of a given value for the plate or tank, respectively.

This transfer function has a LOOCV of 2.25 an adjusted R² of 0.77, but most importantly, the RMSE on the test set drops to 4.36.

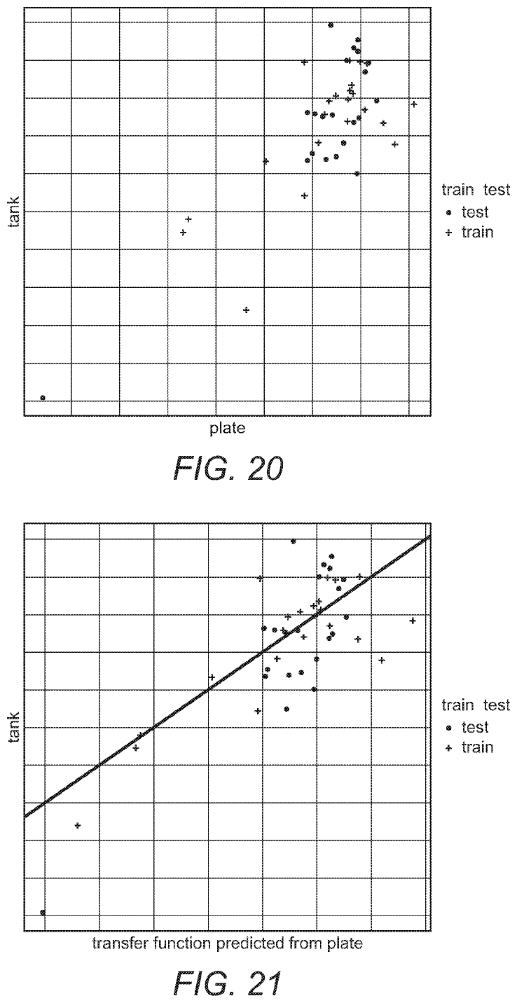

After getting more data and updating the plate and tank data, the plate vs. tank values for the primary metric of interest are as shown in FIG. 20.

The simple linear model tank=b+m₁*plate_value₁, where b=2.735544, m₁=0.009768, had mixed results for these data. The LOOCV is 3.16 and the adjusted R² is 0.49. The LOOCV is worse and the adjusted R² much worse than the previous iteration, but the RMSE on the test set goes down significantly to 2.8.

The prediction engine was run with a weighted least squares model of the form above: tank=b+m₁*plate_value₁+m₂*plate_value₁*plate_value₂, but with regression coefficients mᵢ dependent upon the number of replicates at tank scale, where b=6.996, m1=0.01876, and m2=0.000237 with the same two polyketides (as before in mg/L). Here, an improved model was obtained by all metrics except the LOOCV, as shown in FIG. 21. (The plate values were provided by a statistical plate model.) These statistics are LOOCV=3.14, adjusted R\hat{ }2=0.79, and RMSE on the test set=2.99. As background to factoring the number of tank-scale replicates into the weights mᵢ, the weight vector is determined using ordinary least squares by solving y=Xm+e (here y is a vector of the observed tank values and X is a matrix of the plate values). The weight vector is computed as m=(X^{T}X)⁻¹X^{T}*y. This formulation assumes the variances of the errors (which are random variables) are all the same. However, this assumption generally does not hold in experiments—the number of replicates in the tanks greatly affects variance calculations, and strains typically do not have equal variances, so their errors in this formulation also will not be equal. Allowing the errors to be different, then when we fit the model above, we instead get m=(X^{T}WX)⁻¹X^{T}Wy where W is a diagonal matrix and the diagonal entries are the “weights”. The weights are interpreted as being wᵢ=1/sigmaᵢ², where sigmaᵢ² is the variance of the i^{th} error. This effectively means that more weight (more influence in the fit too) is given to observations with small variance, and less weight (influence) is given to observations with high variance. According to embodiments of the disclosure, we take wᵢ=the number of tank replicates, and in that way strains that have more observations have more weight in the fit because less error overall is expected in the observations of those strains.

In another trial, the prediction engine produced another prediction (transfer) function, where the time the assays were taken was changed and a new set of training strains was used. There is no test data for this function yet. Using the previous weighted least squares approach for the same polyketides as above with the formula tank=b+m₁*plate_value₂+m₂*plate_value₂*plate_value₃, where b=−4.482, m₁=0.05247, m₂=0.0001994, the adjusted R² jumps to 0.93, but the LOOCV is high at 7.44, suggesting there are some high leverage points.

An additional plate value for this model was tested, still using weighted least squares but using the formula b+m₁*plate_value₂+m₂*plate_value₂*plate_value₃+m₃*plate_value₄, where b=−1.810, m₁=0.0563, m₂=0.0001524, m₃=0.5897, plate_value₂ and plate_value₃ are mg/L metrics for the same two polyketides as above, and plate_value₄ is biomass measured in optical density (OD600). The LOOCV dropped to 6.22, still higher than before, but much lower than the previous value and the adjusted R\hat{ }2 is now 0.95. Of course, the true test of this transfer function is testing its predictive power on new strains.

### Example 2

This second example mirrors some aspects of Example 1 in that a set of transfer functions were fit that successively included additional plate measurements per plate (e.g., different types of measurements such as yield, biomass) to try to fit a finer estimate of tank performance. This Example 2 covers one main product, an amino acid produced by a Corynebacterium. Additionally, this example shows the case of applying the transfer function to a different tank variable measurement (here dubbed “tank_value₂”).

One Tank Measurement, Multiple Plate Measurements

Model 1

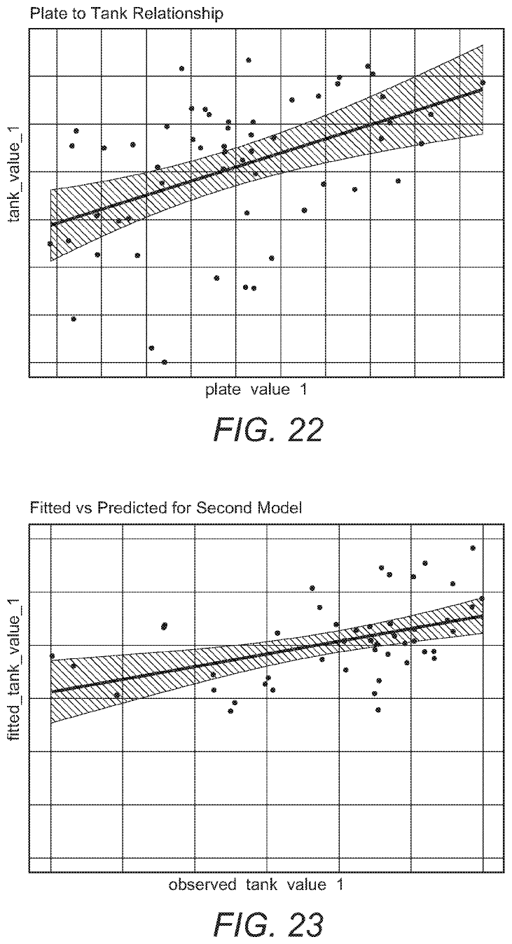

In the first model we fit a simple model that assumed tank_value₁˜1+plate_value₁, according to embodiments of the disclosure. Note that “˜” refers to a “function of, according to a predictive model, such as linear regression or multiple regression.” The underlying plot of FIG. 22 shows the relationship between values of the plate value (represented in the statistical plate model) against the observed tank value.

As can be seen from the plot, when modeling the tank value output on one of the plate metrics, there is potentially a linear relationship between the two.

Taking another step, the prediction engine conducted LOOCV (leave-one-out cross validation) to get the performance of the model by training on every strain except for one, then testing the fit against that one value. The LOOCV score, then, is the average of all the test metrics taken as each data point is removed.

Doing so resulted in the following performance:

| ## | RMSE | MAE |
|---|---|---|
| ## 1 | 3.262872 | 2.532292 |

In particular, with RMSE, the prediction engine computed the ratio of RMSE to the mean tank performance to get a sense of the magnitude of the error relative to the average outcome:

##[1]5.416798

This result indicates that there's about 5% error on the estimate relative to the average values of the tank performance.

Model 2

Now that the inventors had obtained a baseline, they added to the model another measurement from the same plate to compare performance, resulting in a predictive function of the form tank_value₁˜plate_value₁+plate_value₂, with the following statistics:

| ## | RMSE | MAE |
|---|---|---|
| ## 1 | 3.376254 | 2.59808 |

Performance appears slightly worse in this case, as the RMSE and the MAE are a bit higher. See FIG. 23.

Model 3

Finally, in a third example of this process the inventors added yet another factor, such that the model is tank_value₁˜plate_value₁+plate_value₂+plate_value₃.

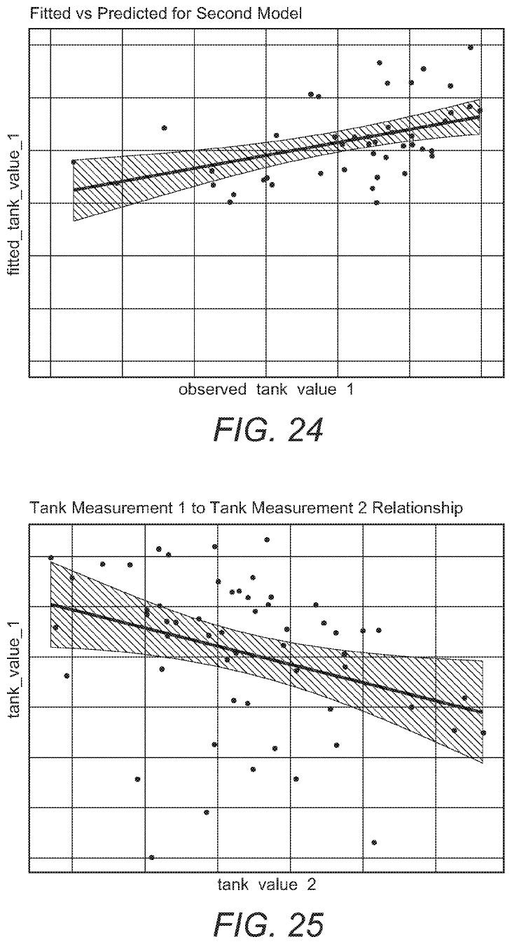

Referring to FIG. 24, this provides a slightly better fit than the first model, as the LOOCV using an RMSE metric is slightly lower for this model.

| ## | RMSE | MAE |
|---|---|---|
| ## 1 | 3.224997 | 2.51152 |

Accordingly the relative percent error is slightly lower than the original model.

##[1]5.353921

Multiple Tank Measurements

As referenced, the transfer function can be applied to predict multiple outcomes for the same tank. For example, the prediction engine fit a model previously of the form tank_value₁˜plate_value₁, but in another trial the prediction engine fit another model to a different output (e.g., yield instead of productivity): tank_value₂˜plate_value₁. FIG. 25 plots two measured tank values against each other.

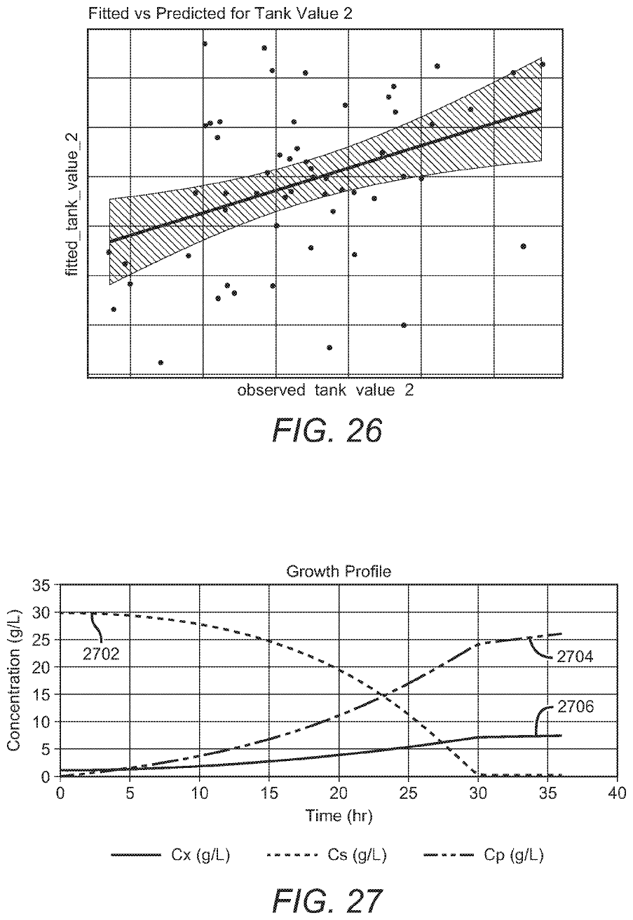

Referring to FIG. 26 the prediction engine fit a model of the form tank_value₂˜plate_value₁, where the observed measurements for tank_value₂ are known a priori to be much more variable than those for tank_value₁. Thus, one would expect that, a priori, the metrics for this model will not be as good as those above. The prediction engine fits this model resulting in an RMSE and MAE of:

| ## | RMSE | MAE |
|---|---|---|
| ## 1 | 0.6315165 | 0.501553 |

Compared the RMSE to the actual value provides a sense of the magnitude of the error:

##[1]19.88434

If desired, the iterative approach may be repeated as described above to add or remove features based on the model's LOOCV performance.

Predictive Model Accounting for Microbial Growth Characteristics

The section “Other statistical models” herein refers to a variety of predictive models. According to embodiments of the disclosure, the prediction engine accounts for microbial growth characteristics. According to embodiments of the disclosure, the prediction engine combines multiple plate-based measurements into a few microbially relevant parameters (e.g., biomass yield, product yield, growth rate, biomass specific sugar uptake rate, biomass specific productivity, volumetric sugar uptake rate, volumetric productivity) for use in transfer functions.

According to embodiments of the disclosure, a transfer function is a mathematical equation that predicts bioreactor performance based on measurements taken in one or more plate-based experiments. According to embodiments of the disclosure, the prediction engine combines the measurements taken in plates into a mathematical equation, e.g.:

PBP=a+b*PM1+c*PM2 . . . n*PMn

in which:
PBP=predicted bioreactor performance (e.g., y in other examples herein),
PMi=the ith plate data variable (e.g., first scale performance data variable xᵢ in other examples herein), which can be a measurement or a function of measurements, such as a combination of measurements or a statistical function of measurements (e.g., a statistical plate model), and
a, b, c, n, may be represented as mᵢ as in other examples herein

The above equation is a linear equation. According to embodiments of the disclosure, the prediction engine may also employ transfer functions of the following form:
quadratic equation (e.g., PBP=a+b*PM1\hat{ }2+c*PM2\hat{ }2)
interaction equation (e.g., PBP=a+b*PM1+c*PM2+d*PM1*PM2)
a combination of different equations

According to embodiments of the disclosure, the prediction engine employs a transfer function that accounts for microbial growth characteristics. Combining linear with quadratic, polynomial or interaction equations can result in many parameters (e.g., a, b, c, d, n) to fit. In particular when only few “ladder strains” (set of diverse strains that have different and known performance) exist against which to calibrate the model, this can result in overfitting of the data and poor predictive value

Thus, based on microbial growth dynamics, the prediction engine may employ a mathematical framework that combines multiple measurements into a few microbially relevant parameters (e.g., biomass yield, product yield, growth rate, biomass specific sugar uptake rate, biomass specific productivity, volumetric sugar uptake rate, volumetric productivity) using selected subtractions, divisions, natural logarithms and multiplications between measurements and parameters. (This approach is discussed further with respect to a prophetic example.)

In general, the prediction engine of embodiments of the disclosure considers two types of plate-based measurements:
Start & end-point measurements, which can be used to assess conversion yields
Mid-point measurements, which can be used to assess conversion rates and yields

Start & End-Point Measurements and Calculation of Microbial Parameters

Typical measurements:

Cx—Biomass concentration (e.g., measured by optical density (“OD”))

Biomass concentration at the start point of the main culture can be either:
Deduced from measuring biomass at the end point in a seed culture, and correcting for transfer volume and main culture volume, i.e., biomass concentration at start point of main culture=biomass concentration at end point of seed culture*(seed to main transfer volume)/(main start volume). A seed culture includes the workflow to revive a set of strains from a frozen condition. The “main” culture includes the workflow to test the performance of the strains.
Estimated as constant from development experiments (e.g., when all strains have a starting biomass concentration of OD 0.1-0.15, the average could be taken as a proxy). The biomass concentration at the end of cultivation (growing a microorganism under particular conditions) is typically much higher than at the start, and the biomass concentration at the start can mathematically be left out of some equations (e.g., if final biomass concentration is more than ten times higher than initial concentration, when measuring biomass yield).

Cp—Product concentration

Note: the same measurements and calculations for product concentration can be performed for byproducts of interest.

Product concentration at start can be either:
Deduced from measuring product at end in seed culture, and correcting for transfer volume and main culture volume, i.e., product concentration at start of main culture=(product concentration at end of seed)*(transfer volume)/(main start volume)
Estimated as constant from development experiments (e.g., when all strains have a starting product concentration of 0.1-0.15 g/L the average could be taken as proxy). Please note that the product concentration at the end of cultivation is typically much higher than at the start, and that the product concentration at the start can mathematically be left out.

Cs—Sugar concentration

Sugar concentration at the start is a known parameter from medium preparation.

Sugar concentration at the end of cultivation is often zero, but can be measured, if needed.

Calculation of microbially relevant parameters:

Biomass yield (Ysx, gram cells per gram sugar)

$$Ysx = \frac{Cx ( end ) - Cx ( start )}{Cs ( start ) - Cs ( end )}$$

i.e., biomass yield=(biomass concentration at end−biomass concentration at start)/(sugar concentration at start−sugar concentration at end)

Product (or byproduct) yield (Ysp, gram product per gram sugar)

$$Ysp = \frac{Cp ( end ) - Cp ( start )}{Cs ( start ) - Cs ( end )}$$

Product (or byproduct) yield=(product concentration at end−product concentration at start)/(sugar concentration at start−sugar concentration at end)

Mid-point measurements & calculation of microbial parameters

Typical measurements:

Time, e.g., t1 and t2

Note: t1 can be start of main cultivation. See above for how to estimate Cx and

Cp at the start of cultivation

Cx—Biomass concentration (e.g. measured by optical density)

According to embodiments of the disclosure, biomass concentration at t1 or t2 is measured, if possible given broth composition

Cp—Product concentration

According to embodiments of the disclosure, product concentration at t1 and t2 is measured

Cs—Sugar concentration

According to embodiments of the disclosure, sugar concentration at t1 or t2 is measured

Sugar concentration at start is a known parameter from medium preparation

Calculations

Biomass yield (Ysx, gram cells per gram sugar)

$$Ysx = \frac{Cx ( t 2 ) - Cx ( t 1 )}{Cs ( t 1 ) - Cs ( t 2 )}$$

i.e., biomass yield=(biomass concentration at t2−biomass concentration at t1)/(sugar concentration at t1−sugar concentration at t2)

Product yield (Ysp, gram product per gram sugar)

$$Ysp = \frac{Cp ( t 2 ) - Cp ( t 2 )}{Cs ( t 1 ) - Cs ( t 2 )}$$

i.e., product yield=(product concentration at t2−product concentration at t1)/(sugar concentration at t1−sugar concentration at t2)

Exponential growth rate (mu, per hour)

$$mu = \frac{ln ( \frac{Cx ( t 2 )}{Cx ( t 1 )} )}{( t 2 - t 1 )}$$

i.e., mu=ln(biomass concentration at t2/biomass concentration at t1)/(time of t2−time of t1)

based on exponential growth: Cx(t2)=Cx(t1)*exp(mu*(t2−t1))

Biomass specific sugar uptake rate (qs, gram sugar per gram cells per hour)

$$qs = \frac{( ln ( \frac{Cx ( t 2 )}{Cx ( t 1 )} ) * ( {Cs}_{} ( t 1 ) - Cs ( t 2 ) )}{( Cx ( t 2 ) - Cx ( t 1 ) ) * ( t 2 - t 1 )}$$

i.e., qs=[ln(biomass concentration at t2/biomass concentration at t1)*(sugar concentration at t1−sugar concentration at t2)]/[(biomass concentration at t2−biomass concentration at t1)*(time t2−time t1)]

based on:

dCx/dt=mu*Cx

dCx/dt=qs*Ysx*Cx

qs=mu/Ysx

Mu=ln(Cx(t2)/Cx(t1))/(t2−t1)

Ysx=(Cx(t2)−Cx(t1)/(Cs(t1)−Cs(t2)

Biomass specific productivity (qp, gram product per gram cells per hour)

$$qp = \frac{( ln ( \frac{Cx ( t 2 )}{Cx ( t 1 )} ) * ( Cp ( t 2 ) - Cp ( t 1 ) )}{( Cx ( t 2 ) - Cx ( t 1 ) ) * ( t 2 - t 1 )}$$

qp=[ln(biomass concentration at t2/biomass concentration at t1)*(product concentration at t2−product concentration at t1)]/[(biomass concentration at t2−biomass concentration at t1)*(time t2−time t1)]

based on:

qp=qs*Ysp

qp=[(mu/biomass yield)]*[(product concentration at t2−product concentration at t1)/(sugar concentration at t1−sugar concentration at t2)]

qp=(ln(biomass concentration at t2/biomass concentration at t1)/(time of t2−time of t1)/[(biomass concentration at t2−biomass concentration at t1)/(sugar concentration at t1−sugar concentration at t2)])*[(product concentration at t2−product concentration at t1)/(sugar concentration at t1−sugar concentration at t2)]

qp=ln(Cxt2/Cxt1)/(t2−t1)/Cxt2−Cxt1/Cst2−Cst1*Cpt2−Cpt1/Cst1−Cst2

Removing Cs's and simplifying to:

qp=ln(Cxt2/Cxt1)/(t2−t1)/((Cxt2−Cxt1)*(Cpt2−Cpt1))

The following parameters Rs and Rp are process rate parameters, distinguished from the above microbe rate parameters (qs and qp). One difference is that a microbe rate parameter is a per-cell metric, whereas a process parameter is a collective rate parameter dependent upon the number of cells (e.g., Rs=qsCx).

Volumetric sugar conversion (Rs, mmol sugar per liter per hour)

$$Rs = \frac{( Cs ( t 1 ) - Cs ( t 2 ) )}{( t 2 - t 1 )}$$

Rs=(sugar concentration at t1−sugar concentration at t2)/(time at t2−time at t1)

Volumetric productivity (Rp, mmol product per liter per hour)

$$Rp = \frac{( Cp ( t 2 ) - Cp ( t 1 ) )}{( t 2 - t 1 )}$$

Rp=(product concentration at t2−product concentration at t1)/(time at t2time at t1)

### Prophetic Example

The following is a prophetic example that accounts for the exponential growth behavior of microbes.

Glucose consumption, biomass formation and product formation were modeled for microbes with a variety of sugar uptake rates, biomass yields and product yields, using the following kinetic growth model formulas:

Biomass-specific sugar uptake rate (qs), dependent on sugar concentration:

qs=qs,max*Cs/(Ks+Cs)

Sugar consumption (dCs) per time interval (dt), dependent on biomass specific sugar uptake rate and biomass concentration, and sugar feed rate:

dCs/dt=−qs*Cx+Fs

Biomass production (dCx) per time interval (dt), dependent on biomass specific sugar uptake rate, sugar dissimilation for maintenance, biomass concentration, and biomass yield:

dCx/dt=qs*Cx*Ysx,max

Product formation (dCx) per time interval (dt), dependent on biomass specific sugar uptake rate, sugar dissimilation for maintenance, biomass concentration, and product yield:

dCx/dt=qs*Cx*Ysp

Some parameters are assigned as follows:

| Parameter | Default value | Unit | Description |
|---|---|---|---|
| Cₓ(0) | 1 | gX/L | Starting biomass concentration |
| Cₛ(0) | 30 | gS/L | Starting sugar concentration |
| Fs | 0.5 | gS/L/h | Sugar feed rate |
| q_{s, max} | 0.4-0.7 | gS/gX/h | Maximum sugar uptake rate |
| Kₛ | 0.5 | gS/L | Affinity value for sugar uptake |
| | | | rate |
| Y_{sx, max} | 0.05-0.15 | gX/gS | Maximum biomass yield |
| Yₛₚ | 0.525-0.675 | gP/gS | Product yield |

Input parameters for the model are variable sugar uptake rate, variable biomass yield (Ysx), variable product yield (Ysp), and some constant parameters.

Table A below shows the variable (maximum) sugar uptake rate (qs) used in hypothetical scenarios A-G:

| | Sugar uptake rate qs |
|---|---|
| Scenario | (g sugar/g cells/h) |
| A | 0.4 |
| B | 0.45 |
| C | 0.5 |
| D | 0.55 |
| E | 0.6 |
| F | 0.65 |
| G | 0.7 |

Table B below shows variable biomass yield (Ysx) and variable product yield (Ysp) (trade-off values) used in hypothetical scenarios 1-9.

| | Biomass yield Ysx | Product yield Ysp |
|---|---|---|
| Scenario | (gX/gS) | (gP/gS) |
| 1 | 0.049286018 | 0.675 |
| 2 | 0.061607522 | 0.65625 |
| 3 | 0.073929026 | 0.6375 |
| 4 | 0.086250531 | 0.61875 |
| 5 | 0.098572035 | 0.6 |
| 6 | 0.11089354 | 0.58125 |
| 7 | 0.123215044 | 0.5625 |
| 8 | 0.135536548 | 0.54375 |
| 9 | 0.147858053 | 0.525 |

Table C below shows constant parameters used for the example:

| parameter | Value | Units |
|---|---|---|
| Initial cell concentration Cx0 | 1 | G cells/L |
| Initial sugar concentration Cs0 | 30 | G sugar/L |
| Sugar feed rate | 0.5 | G Sugar/L/h |
| Sugar uptake affinity constant | 0.5 | G sugar/L |

FIG. 27 plots sugar (Cs) 2702, product (Cp) 2704 and biomass (Cx) 2706 concentrations that were estimated over time using the kinetic growth model. See Table D for an example with a sugar uptake rate of 0.5 g sugar/g cells/h, a biomass yield of 0.1355 g biomass/g sugar, and a product yield of 0.544 g product/g sugar.

As show in Table D below, samples were simulated (including a low level of noise, 0.3%) using the kinetic growth model at different time points for a combination of the different scenarios A-G and 1-9. See below for modeled sugar, product and biomass concentrations after 20 hours of cultivation. The values were compared against the product yield (Ysp-ferm) of the strains in fermentations, which are assumed to be the same as the product yield (Ysp) of the microbe.

TABLE D

| | | | Plate Cs | Plate Cp | Plate Cx | Actual product |
|---|---|---|---|---|---|---|
| Microbe | Microbe | Microbe | after 20 h | after 20 h | after 20 h | yield Ysp in |
| qs | Ysx | Ysp | (g/L), with | (g/L), with | (g/L), with | fermenter (gP/gS), |
| (g/g/h) | (gX/gS) | (gP/gS) | noise | noise | noise | with noise |
| 0.4 | 0.049286018 | 0.675 | 30.540 | 6.489 | 1.469 | 0.678515 |
| 0.4 | 0.061607522 | 0.65625 | 29.923 | 6.670 | 1.622 | 0.663999 |
| 0.4 | 0.073929026 | 0.6375 | 29.314 | 6.800 | 1.792 | 0.637475 |
| 0.4 | 0.086250531 | 0.61875 | 28.902 | 6.938 | 1.971 | 0.616472 |
| 0.4 | 0.098572035 | 0.6 | 28.049 | 7.124 | 2.173 | 0.598028 |
| 0.4 | 0.11089354 | 0.58125 | 27.457 | 7.255 | 2.384 | 0.569804 |
| 0.4 | 0.123215044 | 0.5625 | 26.762 | 7.491 | 2.631 | 0.574604 |
| 0.4 | 0.135536548 | 0.54375 | 25.980 | 7.612 | 2.898 | 0.536564 |
| 0.4 | 0.147858053 | 0.525 | 25.150 | 7.782 | 3.194 | 0.525984 |
| 0.45 | 0.049286018 | 0.675 | 29.121 | 7.481 | 1.539 | 0.667671 |
| 0.45 | 0.061607522 | 0.6565 | 28.401 | 7.715 | 1.711 | 0.654201 |
| 0.45 | 0.073929026 | 0.638 | 27.541 | 7.987 | 1.925 | 0.642866 |
| 0.45 | 0.086250531 | 0.619 | 26.671 | 8.185 | 2.144 | 0.613148 |
| 0.45 | 0.098572035 | 0.6 | 25.874 | 8.462 | 2.390 | 0.605946 |
| 0.45 | 0.11089354 | 0.5815 | 24.933 | 8.693 | 2.659 | 0.587953 |
| 0.45 | 0.123215044 | 0.563 | 24.067 | 9.022 | 2.976 | 0.567682 |
| 0.45 | 0.135536548 | 0.544 | 23.041 | 9.269 | 3.323 | 0.541574 |
| 0.45 | 0.147858053 | 0.525 | 21.858 | 9.563 | 3.689 | 0.530735 |
| 0.5 | 0.049286018 | 0.675 | 27.400 | 8.536 | 1.620 | 0.665161 |
| 0.5 | 0.061607522 | 0.6565 | 26.426 | 8.816 | 1.825 | 0.644647 |
| 0.5 | 0.073929026 | 0.638 | 25.504 | 9.212 | 2.069 | 0.634518 |
| 0.5 | 0.086250531 | 0.619 | 24.611 | 9.538 | 2.322 | 0.618178 |
| 0.5 | 0.098572035 | 0.6 | 23.492 | 9.838 | 2.630 | 0.594583 |
| 0.5 | 0.11089354 | 0.5815 | 22.293 | 10.328 | 2.963 | 0.586114 |
| 0.5 | 0.123215044 | 0.563 | 20.841 | 10.726 | 3.351 | 0.56512 |
| 0.5 | 0.135536548 | 0.544 | 19.592 | 11.146 | 3.774 | 0.540532 |
| 0.5 | 0.147858053 | 0.525 | 18.085 | 11.543 | 4.250 | 0.526556 |
| 0.55 | 0.049286018 | 0.675 | 25.811 | 9.628 | 1.689 | 0.660924 |
| 0.55 | 0.061607522 | 0.6565 | 24.845 | 10.053 | 1.943 | 0.647998 |
| 0.55 | 0.073929026 | 0.638 | 23.641 | 10.513 | 2.216 | 0.638271 |
| 0.55 | 0.086250531 | 0.619 | 22.276 | 11.038 | 2.543 | 0.6244 |
| 0.55 | 0.098572035 | 0.6 | 20.805 | 11.544 | 2.901 | 0.602668 |
| 0.55 | 0.11089354 | 0.5815 | 19.268 | 12.030 | 3.301 | 0.5724 |
| 0.55 | 0.123215044 | 0.563 | 17.623 | 12.634 | 3.756 | 0.548298 |
| 0.55 | 0.135536548 | 0.544 | 15.779 | 13.209 | 4.275 | 0.549351 |
| 0.55 | 0.147858053 | 0.525 | 13.633 | 13.797 | 4.883 | 0.525766 |
| 0.6 | 0.049286018 | 0.675 | 23.957 | 10.765 | 1.783 | 0.673651 |
| 0.6 | 0.061607522 | 0.6565 | 22.841 | 11.396 | 2.059 | 0.658113 |
| 0.6 | 0.073929026 | 0.638 | 21.211 | 11.969 | 2.388 | 0.634771 |
| 0.6 | 0.086250531 | 0.619 | 19.636 | 12.575 | 2.779 | 0.625067 |
| 0.6 | 0.098572035 | 0.6 | 17.886 | 13.249 | 3.189 | 0.591891 |
| 0.6 | 0.11089354 | 0.5815 | 15.870 | 13.935 | 3.680 | 0.586068 |
| 0.6 | 0.123215044 | 0.563 | 13.837 | 14.767 | 4.250 | 0.562263 |
| 0.6 | 0.135536548 | 0.544 | 11.352 | 15.560 | 4.862 | 0.547687 |
| 0.6 | 0.147858053 | 0.525 | 8.725 | 16.352 | 5.639 | 0.520187 |
| 0.65 | 0.049286018 | 0.675 | 22.360 | 11.910 | 1.884 | 0.676242 |
| 0.65 | 0.061607522 | 0.6565 | 20.668 | 12.653 | 2.196 | 0.641914 |
| 0.65 | 0.073929026 | 0.638 | 18.839 | 13.411 | 2.557 | 0.645884 |
| 0.65 | 0.086250531 | 0.619 | 17.013 | 14.407 | 2.988 | 0.623918 |
| 0.65 | 0.098572035 | 0.6 | 14.603 | 15.227 | 3.506 | 0.598114 |
| 0.65 | 0.11089354 | 0.5815 | 12.223 | 16.191 | 4.059 | 0.578762 |
| 0.65 | 0.123215044 | 0.563 | 9.515 | 17.198 | 4.766 | 0.552749 |
| 0.65 | 0.135536548 | 0.544 | 6.504 | 18.231 | 5.515 | 0.54228 |
| 0.65 | 0.147858053 | 0.525 | 3.319 | 19.183 | 6.442 | 0.522942 |
| 0.7 | 0.049286018 | 0.675 | 20.395 | 13.194 | 1.972 | 0.667681 |
| 0.7 | 0.061607522 | 0.6565 | 18.612 | 14.076 | 2.324 | 0.657479 |
| 0.7 | 0.073929026 | 0.638 | 16.273 | 15.152 | 2.737 | 0.640358 |
| 0.7 | 0.086250531 | 0.619 | 13.845 | 16.164 | 3.242 | 0.616917 |
| 0.7 | 0.098572035 | 0.6 | 11.251 | 17.218 | 3.832 | 0.599234 |
| 0.7 | 0.11089354 | 0.5815 | 8.175 | 18.473 | 4.544 | 0.574191 |
| 0.7 | 0.123215044 | 0.563 | 4.897 | 19.759 | 5.335 | 0.562234 |
| 0.7 | 0.135536548 | 0.544 | 1.492 | 20.931 | 6.221 | 0.542419 |
| 0.7 | 0.147858053 | 0.525 | 0.058 | 20.941 | 6.870 | 0.517798 |

Next, correlations were calculated between:

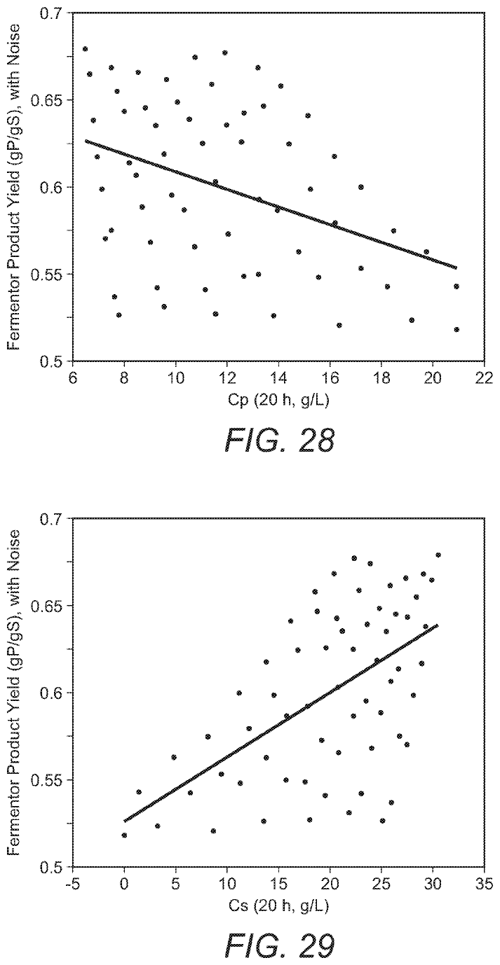

Fermenter yield (key performance indicator (“KPI”) of interest) and Cp after 20 hours in plates (poor correlation), as shown in FIG. 28, resulting in:

| | |
|---|---|
| RSquare | 0.16096 |
| RSquare Adj | 0.147205 |
| Root Mean Square Error | 0.044687 |

Fermenter yield (KPI of interest) and Cs after 20 hours in plates (poor correlation), as shown in FIG. 29, resulting in:

| | |
|---|---|
| RSquare | 0.325469 |
| RSquare Adj | 0.314411 |
| Root Mean Square Error | 0.040068 |

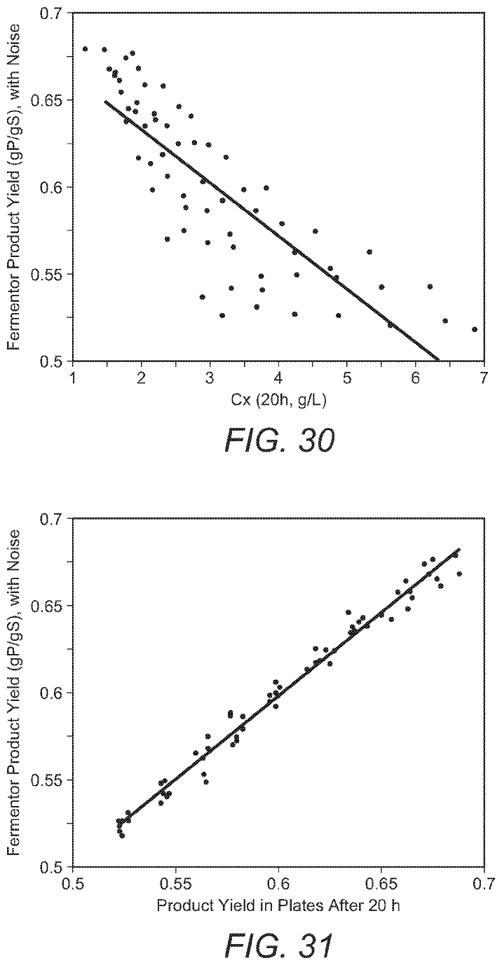

Fermenter yield (KPI of interest) and Cx after 20 hours in plates (poor correlation), as shown in FIG. 30, resulting in:

| | |
|---|---|
| RSquare | 0.678133 |
| RSquare Adj | 0.672857 |
| Root Mean Square Error | 0.027678 |

As shown above, when dealing with a variety of strains with different sugar uptake rates, biomass yields and product yields, and taking a mid-cultivation measurement, individual measurements of sugar, product and biomass do not correlate well with fermenter yield according to this prophetic example.

Statistics were also computed for fermenter (e.g., tank) yield (KPI of interest) and calculation of product yield in plates after 20 hours based on a function (e.g., quotient) of both Cp and Cs after 20 hours in plates, as shown in FIG. 31, resulting in a good correlation:

Ysp=Cp/(Total sugar fed in first 20 h−Cs)

| | |
|---|---|
| RSquare | 0.982442 |
| RSquare Adj | 0.982154 |
| Root Mean Square Error | 0.006464 |

As shown above, estimating product yield by the quotient of (product formed divided by sugar consumed), results in a much better correlation with fermenter yield. This ratio of microbe measurements is an estimate of a microbe property. Other examples of microbe properties are: sugar consumption rate, biomass yield, product yield (Ysp), growth rate, and cell-specific product formation rate.

As noted above, the prediction function may be represented as a weighted sum of variables:

PBP=a+b*PM1+c*PM2 . . . n*PMn

in which:
PBP=predicted bioreactor performance (e.g., y in other examples herein),
PMi=the ith plate data variable (e.g., first scale performance data variable xᵢ in other examples herein), which can be a measurement, or a function of measurements such as a combination of measurements or a statistical function of measurements (e.g., a statistical plate model), and
a, b, c, n, may be represented as mᵢ as in other examples herein

The results of the prophetic example immediately above show that, instead of using measurements such as Cp and Cs directly as the plate data variable PMi, the prediction engine can substitute for PMi one or more microbe properties derived from microbe measurements, such as a quotient or other combination of measurements, according to embodiments of the disclosure.

Transfer Function Development Tool

The transfer function development tool provides a reproducible, robust method for building the transfer function for a given experiment and for recording which strains are removed from the model. Having a development tool for the transfer function relies on the optimization of having a statistical model for predicting performance of lower-throughput performance from higher-throughput performance, and is an optimization in and of itself. Such a product wraps all the optimizations into one package that makes it straightforward for scientists to make use of the transfer function and all its optimizations.

According to embodiments of the disclosure, the raw plate-tank correlation transfer function is reduced to practice in a transfer function development tool (detailed below), along with optimizations such as outlier removal and inclusion of genetic factors. In embodiments of the disclosure, the transfer function development tool may incorporate further optimizations, include other statistical models, modifications to transfer function output, and considerations concerning the plate model.

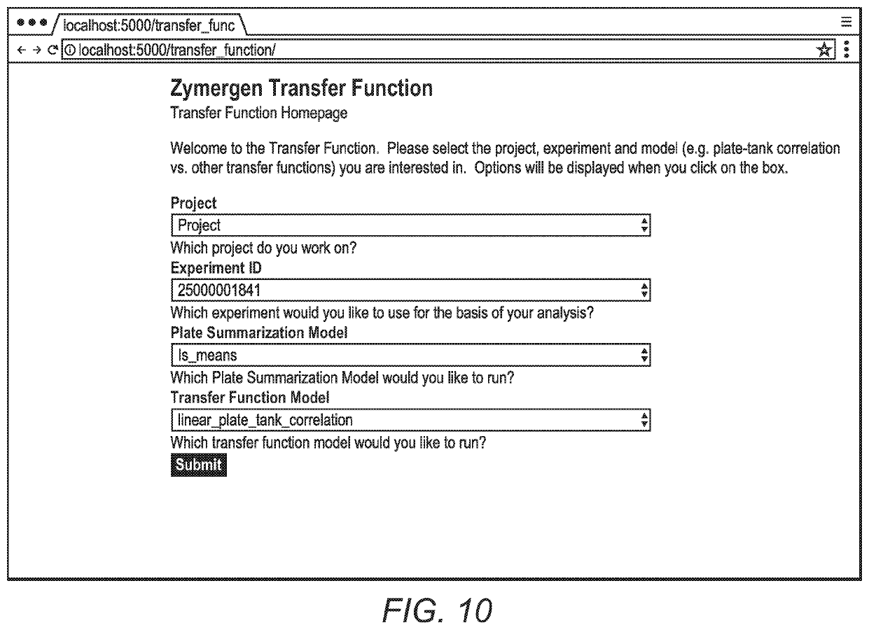

The transfer function development tool, in embodiments of the disclosure, takes high-throughput, smaller-scale performance data for a particular program, experiment, and measurement of interest, learns the appropriate model, and produces predictions for the next scale of work. FIGS. 10-15 show a series of screenshots for an embodiment of the user interface of the tool.

FIG. 10 illustrates a user interface having boxes for user entry of the project name, experiment ID, the selected plate summarization model (here, an LS means model), and the transfer function model to be used (here, a linear regression plate-tank correlation model).

Note the URL line in the address bar 1050 of the graphical user interface. This allows users to follow their progress through the process and confirm they have the correct information for the transfer function they want to implement. This setup is on the front end in the data models, and in the workflow infrastructure.

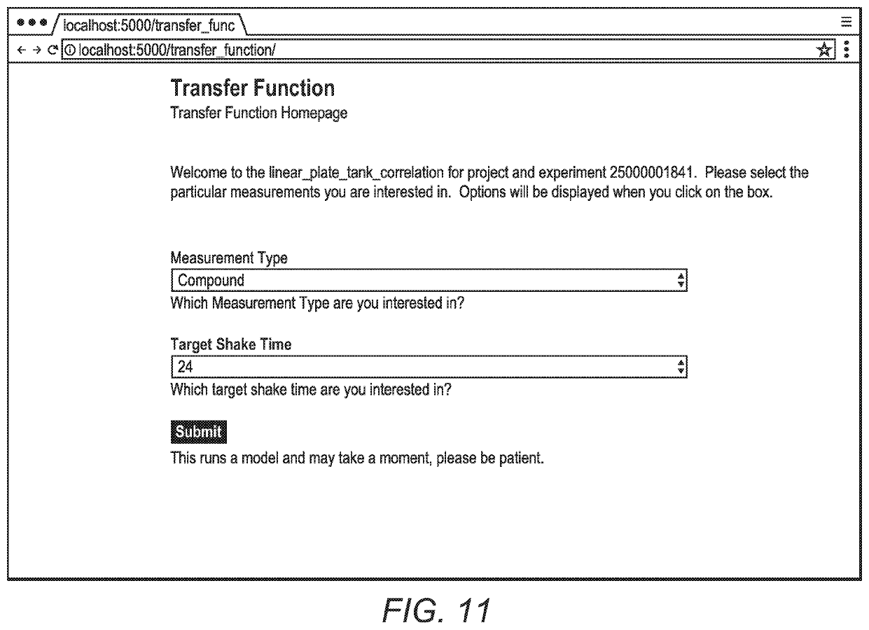

As illustrated in FIG. 11, after users enter their project, experiment, and model selections, they may choose the measurements they are interested in, e.g., amino acid yield (represented by “Compound”) in this example.

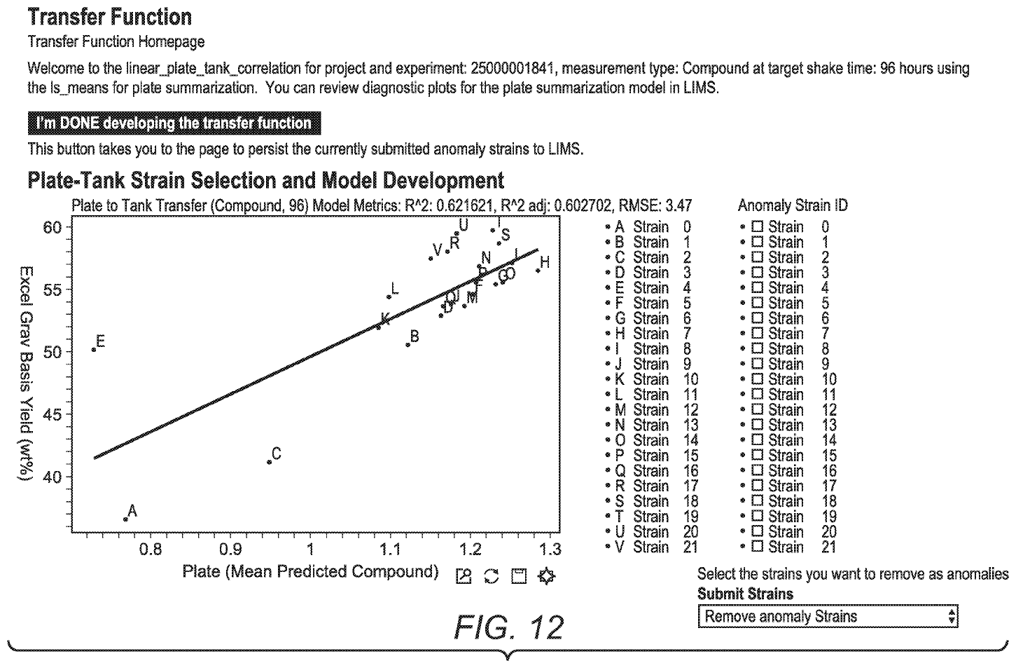

FIG. 12 illustrates a user interface for a plate-tank correlation transfer function after it has been developed for predicting amino acid performance at tank scale, according to embodiments of the disclosure. In this example, the transfer function is a linear fit line. The tool in this figure facilitates outlier evaluation. The user interface provides a list of strains 1202 (“Anomaly Strain ID”), identified by strain ID, along with checkboxes to enable a user to select strains for removal from the transfer function model.

In FIG. 13, the user interface presents ten strains having the highest predicted performance based upon the transfer function with the outliers selected by the user having been removed from the model. Embodiments of the disclosure comprise selecting for manufacture and manufacturing strains in a gene manufacturing system based upon their predicted performance. Such a gene manufacturing system is described in the Codon application—International Application No. PCT/US2017/029725, International Publication No. WO2017189784, filed on Apr. 26, 2017, which claims the benefit of priority to U.S. nonprovisional application Ser. No. 15/140,296, filed on Apr. 27, 2016, all of which are hereby incorporated by reference in their entirety.

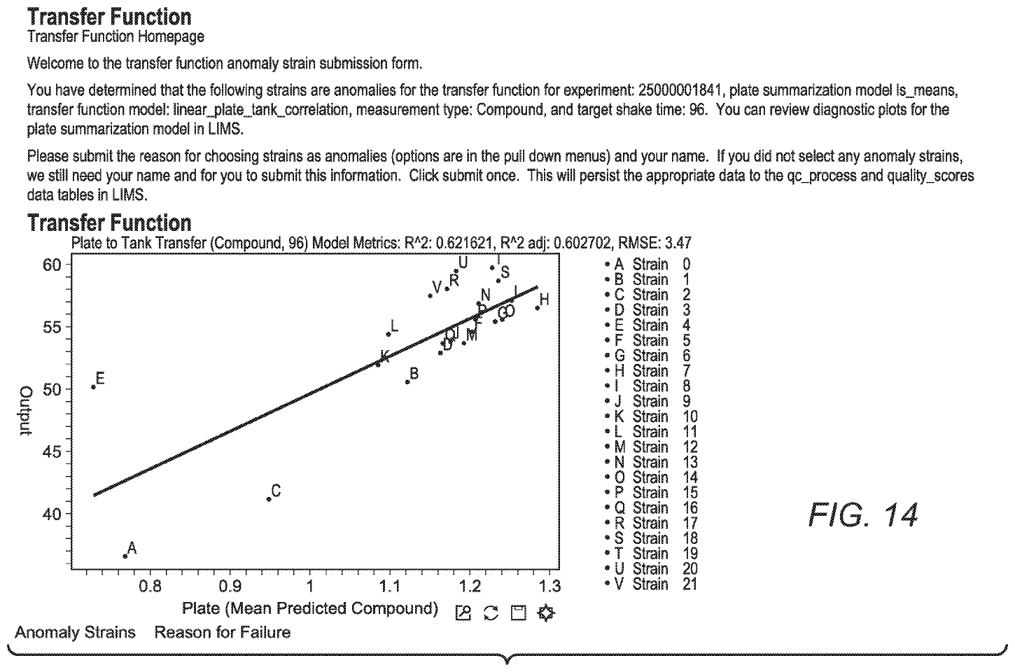

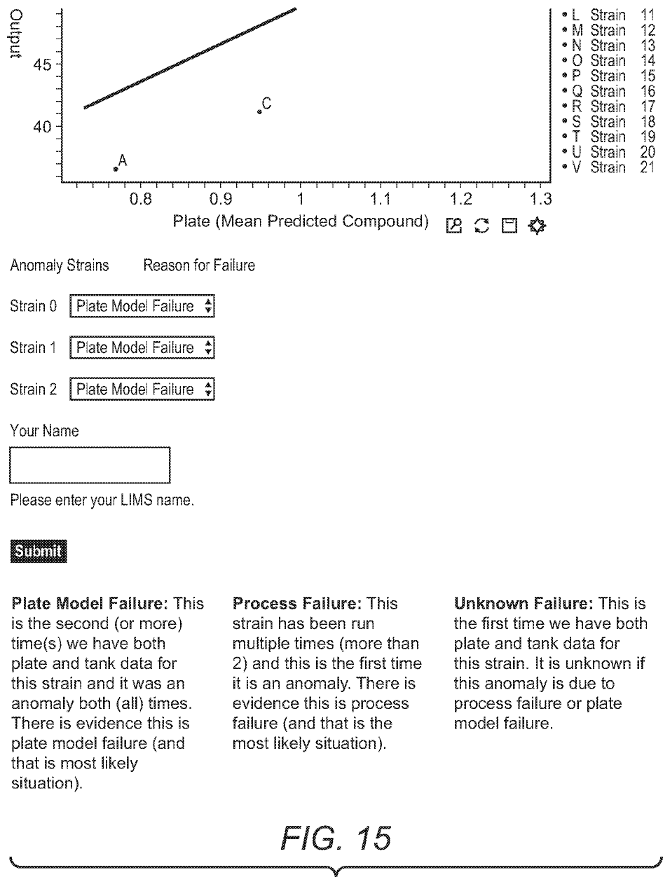

Referring to FIG. 14, the transfer function development tool returns a graphical representation of the chosen transfer function after user-selected outliers have been removed from the model, and (referring to FIG. 15) provides a mechanism to submit quality scores for the removed strains to a database, thus making the final results reproducible and providing a mechanism for users to track strains that are not working well with the existing plate model.

Plate Model Development

According to embodiments of the disclosure, the analysis equipment 214, the prediction engine, or another computer within or outside the LIMS system, whether individually or in any combination (referred to as the “Plate Model engine” or “PM engine” herein), assists in the design of experiments for organisms at a first (plate) scale to generate first-scale performance data used in predicting performance of the organisms at a larger scale.

Embodiments of the disclosure downscale conditions and performance parameters from larger scale (e.g., bench-scale, commercial scale, or both) to smaller (e.g., plate) scale, so that the downscaled parameters may be used to screen organisms at the smaller scale. The PM engine may use the downscaled conditions and parameters to generate first-scale performance data used in predicting performance of the organisms at a larger scale (transfer function). The PM engine may use the predicted larger-scale performance as a factor in the screening of strains, e.g., screen out strains whose predicted larger-scale performance does not satisfy a larger-scale performance threshold.

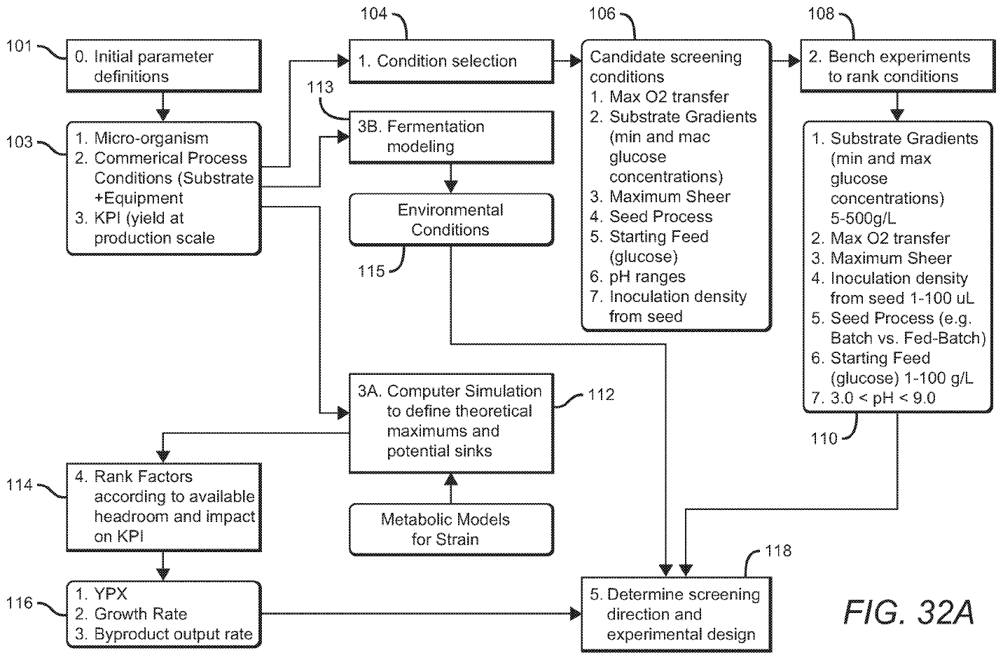

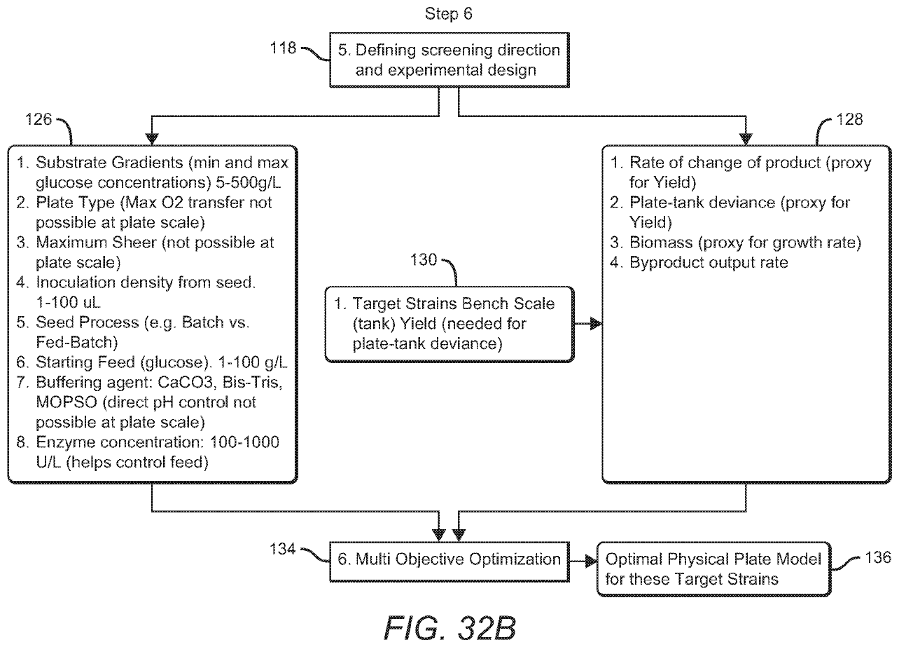

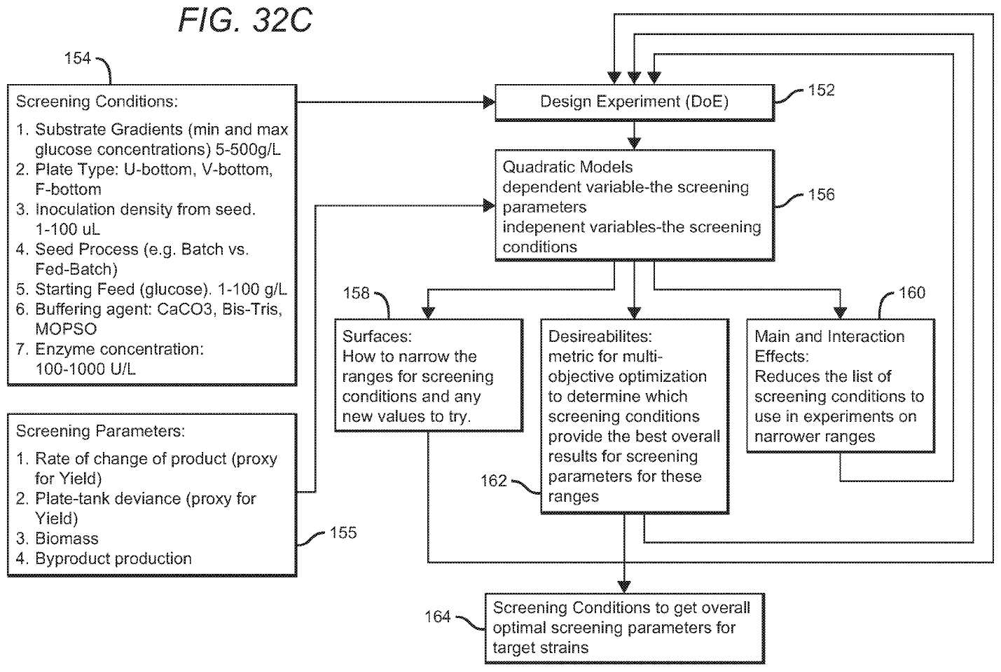

FIGS. 32A and 32B illustrate steps for designing experiments for organisms at a first (plate) scale to generate first-scale performance data used in predicting performance of the organisms at a larger (e.g., bench or commercial) scale. (Note that the steps need not necessarily be performed in the enumerated order, e.g., step 3 can occur before step 2). According to embodiments of the disclosure, the process generally includes:
accessing experimentally determined candidate screening conditions (e.g., output of step 2 below), where the conditions are selected based at least in part upon their contribution to performance parameters (candidate screening parameters) of first strains of an organism (e.g., E. coli) at a second (bench) scale that is larger than a first (e.g., plate) scale;
using, among other things, a computer simulation of the metabolism of the organism (e.g., in step 3A), a computer fermentation model of the organism (at, e.g, bench or commercial scale), or both, to determine candidate first (plate) scale screening parameters, wherein the screening parameters correspond to desired performance of the organism at the second (e.g., bench) scale; and
designing experiments for experimentally determining first-scale performance of second strains of the organism under one or more of the experimentally determined screening conditions or their first-scale proxies, and for screening the second strains based at least in part upon the screening parameters or their first-scale proxies. In some instances, because of the difficulty or impossibility of replicating some screening conditions at the plate level or using the second-scale screening parameters at the plate level, proxies for those conditions or screening parameters, respectively, may be employed. Note that the first and second strains may be the same (type of) organism.

In more detail, according to embodiments of the disclosure, an experimental designer or the PM engine selects candidate conditions that are generally known to affect selected performance parameters (e.g., production of product) of the organism of interest at the second scale. These conditions may include second-scale factors that are not easily physically replicated at the plate scale.

Step 0: As an example, the designer may want to designate as initial parameters for the experiment (101, 103): E. coli as the organism of interest, production of an organic acid product from glucose as the bioprocess, and yield at production (i.e., commercial) scale as a Key Performance Indicator (KPI). Commercial process conditions, such as substrate, fermentation process, and equipment to be used, may also be defined. These definitions may be done at the outset of a project.

Step 1: In this example, the designer may select, as other parameters, candidate screening conditions (104, 106), such as:
Max O2 transfer
Substrate Gradients (minimum to maximum glucose concentrations)
Maximum Sheer (note that sheer is not replicable at plate scale)
Seed Process
Starting Feed (glucose)
Inoculation density from seed
pH

Step 2: Experimentally determine values of performance parameters of different strains of the organism at second (e.g., bench) scale over time in response to different values of the candidate screening conditions defined in step 1. Rank the candidate screening conditions according to the magnitude of their contribution to the performance parameters (including organism viability and the KPI) (108, 110). Contribution to a performance parameter can be determined by varying one candidate screening condition, while holding the others constant. A more efficient technique is to use factorial experimental design and analysis methods known in the art, which are implemented by the PM engine according to embodiments of the disclosure. Based on the experimental response, one can determine preferred ranges for the values of the candidate screening conditions as those ranges resulting in an acceptable range of corresponding performance parameters (e.g., starting feed (glucose) in a range of 1-100 g/L).

For example, in a bench scale fermenter (e.g., between 200 ml and 10 liters) run a series of experiments with different gradients for the candidate screening conditions considered relevant in Step 1, and determine the impact that the different conditions have on the performance parameters at the second scale using known experimental techniques. The performance parameters may relate to the organism itself (e.g., viability, growth rate) and to the product (e.g., yield, biomass). With this information, rank the importance of each second-scale candidate screening condition to each of the second (e.g., bench) scale performance parameters.

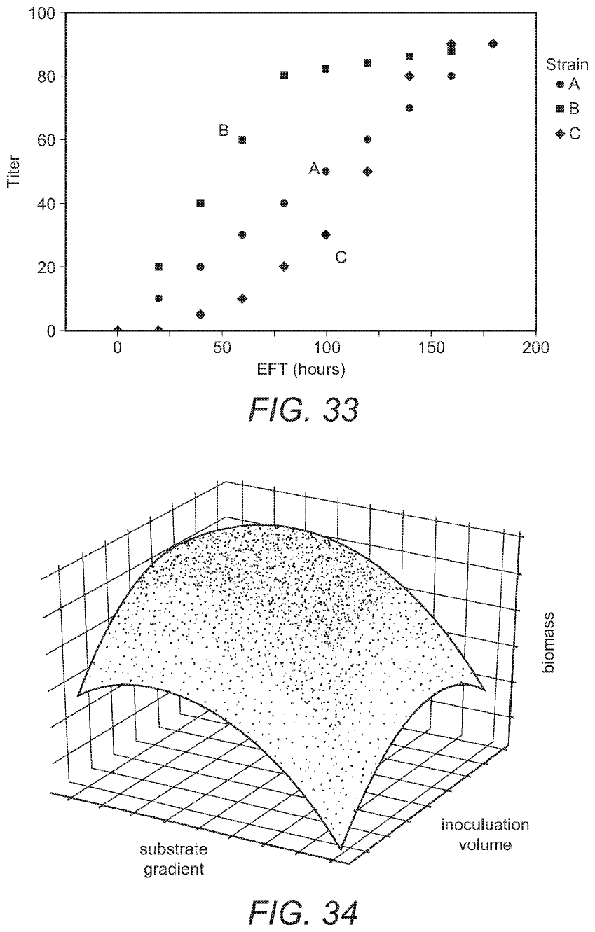

As another example, FIG. 33 illustrates the accumulated titer measured over the course of a bioprocess at different elapsed fermentation times (“EFT”) for three different strains, A, B, and C under the same conditions. These aspects of the fermentation process give insights into the desired screening conditions over different phases of fermentation (e.g., seed and main).

Step 3A: Using a computer simulation-model of the metabolism of the organism, predict maximum theoretical values of performance parameters of different strains of the organism at first (e.g., plate) scale (112). This step determines the theoretical maximum conversion rates from the provided substrate to the desired product, alongside determining potential byproducts (e.g., an undesired organic acid), or limitations (e.g., required presence of certain vitamins or minerals necessary for the organism growth and performance) that could prevent achieving those higher conversion rates.

Metabolic models correlate genes to reaction products for different reaction pathways within a cell. Models such as those provided by the software package COBRApy, employed in embodiments of the disclosure, are widely used for genome-scale modeling of metabolic networks in both prokaryotes and eukaryotes. See A. Ebrahim, COBRApy: COnstraints-Based Reconstruction and Analysis for Python, BMC Systems Biology 2013 7:74, incorporated by reference in its entirety herein. The metabolic pathways in a microbe can be represented by a network of chemical reactions that incorporates the substrate on which it feeds plus other materials it needs to survive, thrive, and grow, such as oxygen, minerals, and vitamins. For more information on metabolic modeling, see, e.g., J. Karr, et al., A Whole-Cell Computational Model, Predicts Phenotype from Genotype, Cell, Vol. 50, Issue 2, pp. 389-401, Jul. 20, 2012, incorporated by reference in its entirety herein.

A bioprocess is defined as the path that connects the substrate (e.g., glucose for E. Coli) to the desired product (a defined organic acid). The conversion of substrate into product is measured by specific yield (as in a single cell). The COBRAPy model can predict the theoretical maximum for that conversion, therefore enabling computation of the headroom for improvement in conversion. It can also provide potential sinks for the substrate or byproducts (e.g., an undesired organic acid) and required substances that may be needed to enable desired reactions (e.g., certain minerals or vitamins).

All this data informs selection of screening directions for the plate experiments, such as measuring the presence of the undesired organic acid to see if the candidate strain has been edited correctly (if a choice is made to block that path to augment the desired organic acid path).

Referring to the example of FIG. 32A, E coli has a known metabolic path from glucose to product, from which one can determine theoretical maximum product performance (e.g., yield). In the example shown, the performance parameters may include the following:
Growth Rate
Viability
Specific Productivity (on a cell level)
YPX (Yield Product Per Biomass)
Byproduct Output rate

These performance parameters are known in the industry to influence the KPI (e.g., yield (grams product/gram substrate) in this case). The byproduct output rate represents non-desirable/negative attributes, e.g., chemicals toxic to the organism or other undesired byproducts. One would want to screen out strains that have a byproduct output rate that is unacceptably high or that have low tolerance to the product.

Step 3B: Using a mathematical model of the fermentation of the organism (at, e.g. second scale, or at commercial scale larger than bench scale), determine environmental conditions (115) for the fermentation, such as a typical quantity of biomass, expected substrate feed rates, typical operational temperature ranges, expected time required to achieve different stages in the fermentation process, and expected oxygen demand at different stages (113). Fermentation models are known in the industry, and can model reactions that occur when large numbers of cells interact with each other (e.g., at bench or commercial production scale). See, e.g., Driving Innovation Through Bioengineering Solutions, Genomatica (date unknown). The environmental conditions may be input to step 5 as additional screening conditions.

With fermentation modeling, one is looking at the initial, known commercial conditions and yields (see Step 0) to define what is likely reasonable to consider for operational ranges. For instance, if the product is toxic to the organism above certain titers, then the screening direction should favor looking for candidate strains that tolerate higher concentrations of the product. If there are benefits in operating at a higher pH, for instance, then one could include a screening condition that allows determination of candidate strains that work better at the higher pH. All these tasks are oriented toward the ultimate goal of improving the KPI. Additionally, the substrate is rarely provided pure to the fermentation process, and the actual concentrations and how that affects yield is easily modeled here, as well.

Step 4: As noted above, step 2 experimentally determines values of the performance parameters of different strains at second (e.g., bench) scale. In step 4, the PM engine compares experimentally determined performance parameter values with their theoretical maximums. The resulting difference represents the potential performance improvement (“available headroom”) that might be achieved in strain performance by adjusting conditions or modifying their genome. Based on these differences and relationships known in the industry between these performance parameters and the KPI, the PM engine ranks the performance parameters, with the highest ranking going to the performance parameter with the greatest available headroom (114). According to embodiments of the disclosure, this step (114) determines the top-ranked performance parameters as those performance parameters whose rank exceeds a rank threshold, whose potential performance improvement exceeds a performance threshold, or a combination of both (e.g., performance parameters in top three of the ranking having a headroom of at least 10%). The top-ranked parameters are identified as candidate screening parameters (116) that may have the greatest potential impact on KPI. In this example, the PM engine has identified YPX, growth rate, and byproduct output rate as the candidate screening parameters.

Step 5: Determine preliminary screening direction and design preliminary plate-scale experiments (118). Screening direction refers to the screening parameters used in experiments at the plate scale, e.g., select microbes with a high yield in plates, while holding other performance parameters constant. This step determines a preliminary physical plate model. The plate model is a collection of media and process constraints designed to make the values obtained at small-scale in high-throughput (e.g., in 96-well plates) as predictive as possible of the values obtained at large scale. According to embodiments of the disclosure, the physical plate model specifies the organism of interest, the screening parameters, the ranges of screening parameter values, and the conditions under which plate-scale experiments are to be run.

The experiments are designed to screen strains of the organism of interest at the smaller (e.g., plate) scale over ranges of top-ranked screening conditions or their proxies. According to embodiments of the disclosure, the screening process comprises determining the response (by screening parameter, e.g., yield) at the smaller scale of each candidate strain to a range of condition values of the top-ranked conditions to determine if the candidate strain is viable under those conditions and satisfies a performance threshold. In this example, the PM engine assembles together the initial parameters (103), the candidate screening conditions 110, the environmental conditions 115, and the candidate screening parameters 116 to preliminarily designs experiments to screen strains of E. coli for yield and growth rate while producing low quantities of undesired byproducts under the top-ranked conditions of substrate gradient, maximum oxygen transfer, and maximum sheer, and under the environmental conditions 115. Thus, Step 5 assembles a preliminary plate model.

Step 6: FIGS. 32B and 32C illustrate step 6. According to embodiments of the disclosure, the PM engine employs multi-objective optimization (“MOO”) techniques to determine optimized condition values that correspond to optimization over multiple objectives that impact the KPI. At this point, the MOO algorithm (134) has as inputs final second-scale screening parameters that represent the screening conditions and parameters from step 5 along with ranges to explore the screening conditions (126) and parameters (128), respectively, as well as a preliminary plate model and preliminary designs for plate-level experiments.

According to embodiments of the disclosure, the MOO employs response surface methodology, described in greater detail below. The final second-scale screening parameters serve as that basis for optimization objectives for the MOO algorithm. The PM engine uses the MOO algorithm to compute optimum values for the step 2 screening conditions that can be controlled at the plate level or their proxies (which are shown in 126). That is, the MOO computes the conditions that result in an optimum over the multiple final screening parameter objectives. According to embodiments of the disclosure, the PM engine includes the optimum condition values in the physical plate model (136).

As described above, some second-scale conditions determined in step 2 (or (e.g., commercial scale) environmental conditions 115) are impossible or difficult to replicate at the first (e.g., plate) scale. For example, maximum oxygen transfer and maximum sheer (listed in 126) are conditions that cannot be replicated on a 96-well plate. Thus, according to embodiments of the disclosure, the PM engine removes those conditions from consideration by the MOO in step 6. According to embodiments of the disclosure, the PM engine substitutes known plate-scale proxies for those removed conditions for which proxies are known, like the type of plate (e.g., well geometry and dimensions) as a proxy for Max O2 transfer, and shaking speed and time as a proxy for in-tank agitation. Overall, the physical plate model is a first scale representation for the bioreactor at the second scale, and, as such, not all conditions must have a plate-scale proxy, rather the collection of conditions at the first scale (plate) serves as a representation for the second scale. The PM engine incorporates into the physical plate model the proxy conditions along with conditions that can be controlled at the plate scale.

Similarly, one or more of the screening parameters output from step 5 may be impossible or difficult to employ at the first (e.g., plate) scale. Thus, the PM engine may employ proxy screening parameters. In this example, yield (e.g., number of grams of organic acid per gram of sugar) cannot be used to screen at plate scale. Thus, the PM engine may instead employ plate-level proxies for yield, such as rate of change of product and plate-tank deviance (128). As shown, the PM engine may also employ biomass as a proxy for growth rate.

The use of proxies at smaller (e.g., plate) scale as surrogates for at least some conditions and performance parameters at larger scale is known in the industry. However, the inventors believe that use of plate-tank deviance, according to embodiments of the disclosure, is novel.

To determine the plate-tank deviance proxy screening parameter, the PM engine knows the second-scale (e.g., bench tank) yield as a reference (130). Plate-tank deviance is a metric developed by the inventors. It measures the absolute value of the difference between a microbe's product performance in a plate (e.g., plate-level titer) and its product performance in a tank (e.g., tank-level yield and productivity). A deviation of 0 indicates perfect agreement between the observed performance in the plate and in the tank. The plate-tank deviance captures, in a single metric, the accuracy of statements such as “this strain performed X % better than its parent in both plates and tanks.” For example, if the deviance is 0 then this statement is perfectly true. As the deviance increases we observe more error. Since we use the absolute value of the difference in performance, the plate-tank deviance is always greater than or equal to 0 and therefore the optimization target is minimization.

Unlike the statistical plant-tank correlation R² between measured organism performance at the plate level vs. measured organism performance in the tank, plate-tank deviance may use bootstrapping, which results in better estimates of the distribution of plate and tank values and measures the relation between those distributions.

Second, it is advantageous to design physical plate models that generalize to many strains. Thus, the modeling/optimization approach should use the per strain information we have. The R² of the plate-tank correlation is a per-plate model metric, whereas all of our other targets are per strain per plate model. Thus, if we wanted to use the R² of the plate-tank correlation as an optimization target, we would have to summarize all of the other responses to the per plate model level and the response surface models would be fit on these summary statistics, losing critical strain information. As a result, the desirability and other model information would not account for per strain variation, thus reducing statistical power and likely leading to poor generalization. By using the plate-tank deviance we have a plate-tank measurement that is compatible with our other objectives and we are able to build the models and desirability functions accounting for strain differences.

According to embodiments of the disclosure, computation of plate-tank deviance may depend upon plate titer and tank yield. Since plate titer and tank yield are on different scales, the PM engine cannot simply compute the difference in values. Further, the PM engine directly compares a single tank value with a particular plate value as there are both more plate values than tank values, and these assays are separated in time. While the PM engine could use the mean for each strain, this hides variability. Therefore, the plate-tank deviance may be computed as follows:
1. Standardize the plate and tank values (e.g. subtract the mean and divide by the standard deviation).
2. Using known statistical techniques, bootstrap plate and tank samples for each strain to estimate the distribution of plate to tank values for each strain.
3. Compute the absolute difference between the plate and tank values.

According to embodiments of the disclosure, the PM engine may also compute a per-strain mean for the plate-tank deviance.

Step 7: According to embodiments of the disclosure, the PM engine uses a statistical plate model as input to a transfer function to predict performance of the strains of interest at the second scale. According to embodiments of the disclosure, the PM engine generates a first-scale statistical model based upon the first-scale physical model, as described in the Transfer Function application. The MOO of Step 6 provides the optimum screening condition values corresponding to the optimum screening parameters. The PM engine uses this data to run experiments using the physical plate model parameters for the strains to determine the statistical plate model. The PM engine may employ the statistical plate model to generate plate-scale performance values as inputs to the transfer function, as described elsewhere herein. The transfer function then predicts performance of the strains at the second (e.g., bench) scale.

Step 8: According to embodiments of the disclosure, the PM engine then selects strains having a predicted second-scale performance exceeding a performance threshold. These strains may serve as base strains for further laboratory experiments in which the base strains' genomes are genetically perturbed. Using these new perturbed strains, the PM engine may repeat steps 2-8 for the perturbed strains until a desired predicted second-scale performance is achieved or an external parameter (e.g., number of iterations) is satisfied. The final physical plate model for the perturbed strains in each iteration is deemed the optimal model (136).

Multi-Objective Optimization Using Response Surface Methodology (RSM)

RSM is an approach to optimizing parameters in complex systems, where the number of parameters and values for those parameters is very large, making exhaustive testing of all possible combinations intractable. RSM supports:
Efficient parameter exploration: By combining quadratic models with optimization, enables exploration of the effect of parameter values not tested in plate model experiments.
Supports sequential experimental design: The information the modeling provides makes it easy to use the results from one experiment to more effectively design the next experiment to hone in on the “optimal” plate model, but can also be used in the context of parallel experiments (e.g., one is started before the other is completed). FIG. 32C illustrates the feedback of results to DoE 152 from blocks 158, 160 and 162.
Easy workflow with good statistical support: It is a well-established (good scientific and theoretical support) and easily implemented workflow, saving a great deal of computing time.
Supports multi-objective optimization: The approach embodiments of the disclosure to using RSM for multi-objective optimization goes beyond finding multiple Pareto optima by providing a ranking metric. A Pareto optimum in this context is a set of plate model parameters such that it is impossible to change any one of those parameters so as to make any one response target better (according to the optimization goals) without making at least one other target worse.
Provides effects estimates: Using optimal designs of experiments (“DoEs”) that support quadratic models allows embodiments of the disclosure to estimate both main, interaction, and polynomial effects. Understanding the effects of the screening conditions on the screening performance parameters supports efficient sequential experimental design. For example, if a parameter has little effect, the PM engine can eliminate it from further investigation. An example of the form of a quadratic equation that is used in embodiments of the disclosure is listed below.

$$response = {\underbrace{{parameter}_{1} + {parameter}_{2}}}_{Main effects} + {( \underbrace{{parameter}_{1} \times {parameter}_{2}} )}_{Interaction effects } + {\underbrace{{parameter}_{1}^{2} + {parameter}_{2}^{2}}}_{Polynomial effects }$$

RSM is one of several possible approaches to standardizing and improving the information/time/money ratio in plate model development. Other methodologies that may be employed are black-box optimization ideas like those in D. Golovin, et al., Google Vizier: A Service for Black-Box Optimization, Google Research, KDD '17, Proceedings of the 23rd ACM SIGKDD International Conference on Knowledge Discovery and Data Mining, pp. 1487-1495 (2017).

Desirability: Multi-Objective Optimization

One approach to RSM supports multi-objective optimization through the use of a desirability metric. The desirability function incorporates response target information, the relative importance of those targets, and response surface models to provide a single metric that ranks the sets of experimental parameter values. Higher desirability means the experimental parameters lead to responses that more closely hit the targets (see below).

According to embodiments of the disclosure, the overall desirability is a weighted geometric mean, D=d₁^{w}^{₁} d₂^{w}^{₂} . . . dₖ^{w}^{ₖ}, where each d_i is a single desirability for a single screening parameter as defined below, and each wᵢ is the importance of the corresponding screening performance parameter as determined by Step 5. For examples regarding importance see Table 2.

According to embodiments of the disclosure, there are three possible desirability functions used for the d_i in the formula above—one each for screening parameters for which maximization is desired, for which minimization is desired, and for which a target value is desired. A reference for these desirability functions is: Derringer, G., and Suich, R. (1980). “Simultaneous Optimization of Several Response Variables.” Journal of Quality Technology 12.4:214-219, incorporated by reference in its entirety herein. According to embodiments of the disclosure, the PM engine employs JMP desirability functions and a JMP profiler to compute those desirabilities for fit models. To use JMP, the PM engine provides the “importance” weights wᵢ of the screening parameter (aka objective). As a reference, please see JMP® 14 Profilers, Version 14, SAS Institute Inc. 2018, incorporated by reference in its entirety herein.

In step 5, the PM engine also provides low, middle and high values (the “three levels”) for the screening parameter objective, along with how individually “desirable” those values are. These individual desirabilities specify the desirability of screening parameter values that fall between these low, high and target values, and how quickly the desirability functions become zero outside of the low and high values. Example values are provided in Table 2 below.

For rate of change of product titer, 0 is the minimum acceptable value, meaning that the amount of product titer should not go down over time. For the desirability set to 0.1, 0 rate of change has low desirability and values below 0 have 0 desirability. Setting the desirability of the middle and high value both at 0.9 indicates that all values between 2 and 4 are equally highly desirable. Similarly, for biomass, a biomass of 6 is not as desirable as 4 or values between 4 and 6, and values larger than 6 should smoothly drop to desirability 0 as per how JMP builds the functions. The PM engine generates the data shown in Table 2 as part of Step 5.

RSM employs desirabilities to compute a multi-objective optimum. As shown in Table 2 below, the desirabilities specify, for each plate-level screening parameter (objective), target ranges, the goal with respect to the target ranges, and weighting to be accorded each target range.

TABLE 2
Desirabilities

| Screening | | Low | Middle | High | Weight |
|---|---|---|---|---|---|
| Performance | | (value, | (value, | (value, | (aka |
| Parameter | Goal | desirability) | desirability) | desirability) | importance) |
| Rate of | Match | (0, 0.1) | (2, 0.9) | (4, 0.9) | 1 |
| change of | Target | | | | |
| product titer | | | | | |
| Plate-tank | Minimize | (0, 1) | (0.1, 0.8) | (0.3, 0.2) | 0.75 |
| deviance | | | | | |
| Biomass | Match | (0, 0.1) | (4, 0.9) | (6, 0.5) | 0.75 |
| | Target | | | | |
| Undesired | Minimize | (0, 1) | (0.05, 0.5) | (0.1, 0.1) | 0.5 |
| byproducts | | | | | |

The PM engine scales the weights to sum to 1. In this example, plate-tank deviance is considered to be ¾ as important the rate of change of titer. And rate of change of glucose ½ as important.

Having chosen RSM as the analytical MOO methodology in embodiments of the disclosure, experiments were designed to support that approach—in particular, D-optimal experimental designs that support a quadratic regression model for each response, while avoiding biased or aliased parameters. At a high level, using a D-optimal design means using the fewest possible variable combinations required to estimate the quadratic models where the conditions are the independent variables and the screening parameters are the dependent variables with high statistical power.

RSM is a workflow and requires several steps as illustrated in FIG. 32C. According to embodiments, the first step is 152, designing an experiment at the first scale. That design is d-optimal for a quadratic model, such as Equation 2 below (weighting coefficients omitted for clarity and usingonly a subset of the screening conditions in 154):

biomass=substrate gradient+plate type+inoculation density+(substrate gradient×plate type)+(substrate gradient×inoculation density)+(plate type×inoculation density)+(substrate gradient)²+(inoculation density)² Equation 2

According to embodiments of the disclosure, the PM engine then causes the robotic lab equipment to conduct the designed experiment at the first scale, determines the resulting performance parameters, which may be deemed screening parameters within the MOO algorithm (155). The next step in RSM is to fit the quadratic models (156), that is, to find the weighting coefficients in models like that in Equation 2.

This approach allows modeling and interpolating how a screening parameter such as biomass is affected across many more variables than those tested (158). Thus, extensive or exhaustive experimentation is avoided. FIG. 34 illustrates a surface shape showing how biomass is modeled and values are interpolated for the biomass response for the batch feeding scheme. The example uses the screening conditions in block 154. The figure shows two screening conditions (dependent variables), inoculation volume and substrate gradients. As seen in the figure, the PM engine can infer optimum values for the screening parameter biomass for values of the screening conditions that were not necessarily tested in the experiment.

According to embodiments of the disclosure, the quadratic models are used to infer values of the screening parameters across the full grid of values in the ranges in 154, which allows using all of these values in the desirability functions described above, giving an overall desirability metric (164) for all screening condition combinations in the grid in 154 while only having experimentally tested those in the d-optimal design (152).

Combining the overall desirability with the main and interaction effects (using standard statistical techniques to get these from the fit models) (160) and surface shapes (158) shows how to narrow the number of screening conditions both in number and in their ranges for the next round of experimentation. This step is a known part of RSM.

In experiments, RSM workflow met screening parameter targets (Table 2) within only three experiments. In one experiment, most of the plate conditions were not meeting both the byproduct and biomass requirements. By the third experiment, most of the strains had a strong R² correlation between predicted and actual second-scale performance, as well as high desirabilities.

The final plate model chosen was one of the two plate models with the highest desirability over all models tested in the final experiment. The conditions in these plate models were reproducible as both of these plate models had high desirabilities in a previous experiment as well. Experiments completed as part of steps 1-5 meant that we started this example RSM with a plate model that had with desirability 0.23 and the final desirability was 0.79.

Machine Learning

Embodiments of the disclosure may apply machine learning (“ML”) techniques to learn the relationship between the given parameters (features) and observed outcomes (e.g., experimental data concerning molecule or material properties). In this framework, embodiments may use standard ML models, e.g. Decision Trees, to determine feature importance. In general, machine learning may be described as the optimization of performance criteria, e.g., parameters, techniques or other features, in the performance of an informational task (such as classification or regression) using a limited number of examples of labeled data, and then performing the same task on unknown data. In supervised machine learning such as an approach employing linear regression, the machine (e.g., a computing device) learns, for example, by identifying patterns, categories, statistical relationships, or other attributes exhibited by training data. The result of the learning is then used to predict whether new data will exhibit the same patterns, categories, statistical relationships or other attributes.

Embodiments of this disclosure may employ unsupervised machine learning. Alternatively, some embodiments may employ semi-supervised machine learning, using a small amount of labeled data and a large amount of unlabeled data. Embodiments may also employ feature selection to select the subset of the most relevant features to optimize performance of the machine learning model. Depending upon the type of machine learning approach selected, as alternatives or in addition to linear regression, embodiments may employ for example, logistic regression, neural networks, support vector machines (SVMs), decision trees, hidden Markov models, Bayesian networks, Gram Schmidt, reinforcement-based learning, cluster-based learning including hierarchical clustering, genetic algorithms, and any other suitable learning machines known in the art. In particular, embodiments may employ logistic regression to provide probabilities of classification along with the classifications themselves. See, e.g., Shevade, A simple and efficient algorithm for gene selection using sparse logistic regression, Bioinformatics, Vol. 19, No. 17 2003, pp. 2246-2253, Leng, et al., Classification using functional data analysis for temporal gene expression data, Bioinformatics, Vol. 22, No. 1, Oxford University Press (2006), pp. 68-76, all of which are incorporated by reference in their entirety herein.

Embodiments may employ graphics processing unit (GPU) or Tensor processing units (TPU) accelerated architectures that have found increasing popularity in performing machine learning tasks, particularly in the form known as deep neural networks (DNN). Embodiments of the disclosure may employ GPU-based machine learning, such as that described in GPU-Based Deep Learning Inference: A Performance and Power Analysis, NVidia Whitepaper, November 2015, Dahl, et al., Multi-task Neural Networks for QSAR Predictions, Dept. of Computer Science, Univ. of Toronto, June 2014 (arXiv:1406.1231 [stat.ML]), all of which are incorporated by reference in their entirety herein. Machine learning techniques applicable to embodiments of the disclosure may also be found in, among other references, Libbrecht, et al., Machine learning applications in genetics and genomics, Nature Reviews: Genetics, Vol. 16, June 2015, Kashyap, et al., Big Data Analytics in Bioinformatics: A Machine Learning Perspective, Journal of Latex Class Files, Vol. 13, No. 9, September 2014, Prompramote, et al., Machine Learning in Bioinformatics, Chapter 5 of Bioinformatics Technologies, pp. 117-153, Springer Berlin Heidelberg 2005, all of which are incorporated by reference in their entirety herein.

Computing Environment

FIG. 16 illustrates a cloud computing environment according to embodiments of the disclosure. In embodiments of the disclosure, software 1010 may be implemented for the prediction engine, the PM engine, the analysis equipment 214 or other computer operations disclosed herein in a cloud computing system 1002, to enable multiple users to generate and apply the transfer function, develop the physical and statistical plate models, control automated laboratory experiments, and perform other computer-implemented operations according to embodiments of the present disclosure. Client computers 1006, such as those illustrated in FIG. 17, access the system via a network 1008, such as the Internet. The system may employ one or more computing systems using one or more processors, of the type illustrated in FIG. 17. The cloud computing system itself includes a network interface 1012 to interface the software 1010 to the client computers 1006 via the network 1008. The network interface 1012 may include an application programming interface (API) to enable client applications at the client computers 1006 to access the system software 1010.

A software as a service (SaaS) software module 1014 offers the system software 1010 as a service to the client computers 1006. A cloud management module 10110 manages access to the system 1010 by the client computers 1006. The cloud management module 1016 may enable a cloud architecture that employs multitenant applications, virtualization or other architectures known in the art to serve multiple users.

FIG. 17 illustrates an example of a computer system 1100 that may be used to execute program code stored in a non-transitory computer readable medium (e.g., memory) in accordance with embodiments of the disclosure. The computer system includes an input/output subsystem 1102, which may be used to interface with human users and/or other computer systems depending upon the application. The I/O subsystem 1102 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the prediction engine may be implemented with a computer system like that of computer system 1100.

Program code may be stored in non-transitory media such as persistent storage in secondary memory 1110 or main memory 1108 or both. Main memory 1108 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 1104 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 1104. The processor(s) 1104 may include graphics processing units (GPUs) for handling computationally intensive tasks.

The processor(s) 1104 may communicate with external networks via one or more communications interfaces 1107, such as a network interface card, WiFi transceiver, etc. A bus 1105 communicatively couples the I/O subsystem 1102, the processor(s) 1104, peripheral devices 1106, communications interfaces 1107, memory 1108, and persistent storage 1110. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 1100. In particular, elements of the LIMS system, the prediction engine, the PM engine, the analysis equipment 214, and any other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 16.

Those skilled in the art will recognize that, in some embodiments, some of the operations described herein may be performed by human implementation, or through a combination of automated and manual means. When an operation is not fully automated, appropriate components of the the LIMS system, the prediction engine, the PM engine, the analysis equipment 214 may, for example, receive the results of human performance of the operations rather than generate results through its own operational capabilities.

Although the disclosure may not expressly disclose that some embodiments or features described herein may be combined with other embodiments or features described herein, this disclosure should be read to describe any such combinations that would be practicable by one of ordinary skill in the art. Unless otherwise indicated herein, the term “include” shall mean “include, without limitation,” and the term “or” shall mean non-exclusive “or” in the manner of “and/or.”

Those skilled in the art will recognize that, in some embodiments, some of the operations described herein may be performed by human implementation, or through a combination of automated and manual means. When an operation is not fully automated, appropriate components of embodiments of the disclosure may, for example, receive the results of human performance of the operations rather than generate results through its own operational capabilities.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world, or that they are disclose essential matter.

In the claims below, a claim n reciting “any one of the preceding claims starting with claim x,” shall refer to any one of the claims starting with claim x and ending with the immediately preceding claim (claim n−1). For example, claim 35 reciting “The system of any one of the preceding claims starting with claim 28” refers to the system of any one of claims 28-34.

### SELECTED EMBODIMENTS OF THE DISCLOSURE

Each embodiment below corresponds to one or more embodiments of the disclosure. Dependencies below are understood to refer back to embodiments within the same set.

### Method Embodiments

Set 1
1. A computer-implemented method of designing experiments for organisms at a first scale to generate first-scale performance data used in predicting performance of the organisms at a larger, second scale, the method comprising:
   a. determining first-scale screening conditions based at least in part upon contribution of second-scale conditions to performance parameters of first strains of an organism at the second scale, wherein the first-scale screening conditions include one or more proxies for second-scale conditions that cannot be replicated at first scale;
   b. determining first-scale screening parameters based at least in part upon computer modeling of the metabolism of the organism at the second scale; and
   c. designing experiments for experimentally screening second strains of the organism under the first-scale screening conditions based at least in part upon the first-scale screening parameters.
2. The method of embodiment 1, further comprising generating a first-scale statistical model of first-scale performance of the second strains, and using the first-scale statistical model to predict performance of the second strains at a third scale.
3. The method of embodiment 2, wherein the third scale is the same as the second scale.
4. The method of any one of embodiments 2 or 3, wherein designing experiments includes screening the second strains based at least in part upon the predicted third-scale performance of the second strains.
5. The method of any one of the preceding embodiments, wherein determining first-scale screening conditions is further based at least in part upon environmental conditions determined from fermentation modeling.
6. The method of any one of the preceding embodiments, wherein determining first-scale screening conditions is further based at least in part upon environmental conditions determined from fermentation modeling of the organism at a third scale larger than the second scale.
7. The method of any one of the preceding embodiments, wherein the first scale is at the scale of a plate, and the second scale is at the scale of a bench tank.
8. The method of any one of the preceding embodiments, wherein the first scale is at the scale of a plate comprising wells wherein each well has a volume within a range of 50-200 microliters, and the second scale is at the scale of a bench tank has a volume within a range of 200 ml-10 liters.
9. The method of any one of the preceding embodiments, wherein determining first-scale screening parameters comprises determining second-scale performance parameters that contribute to a key performance indicator (“KPI”) above a contribution threshold.
10. The method of any one of the preceding embodiments, wherein determining first-scale screening parameters comprises determining second-scale performance parameters based on their potential for improving performance of a KPI.
11. The method of any one of the preceding embodiments, further comprising determining optimum values of the first-scale screening conditions that optimize the first-scale screening parameters collectively at the first scale, wherein designing experiments comprises designing experiments to experimentally determine first-scale performance of the second strains in response to a range of screening condition values around the optimum screening condition values.
12. The method of any one of the preceding embodiments, further comprising determining optimum values of the first-scale screening conditions that optimize the first-scale screening parameters and a plate-tank deviance collectively at the second scale, wherein designing experiments comprises designing experiments to experimentally determine first-scale performance of the second strains in response to a range of screening condition values around the optimum condition values.
13. The method of any one of the preceding embodiments, further comprising controlling performance of experiments to screen the second strains at the first scale using the first-scale screening conditions and the first scale screening parameters.
14. The method of any one of the preceding embodiments, wherein the first strains and the second strains are the same.

### System Embodiments

Set 1
1. A system for designing experiments for organisms at a first scale to generate first-scale performance data used in predicting performance of the organisms at a larger, second scale, the system comprising:
   one or more processors; and
   one or more memories storing instructions, that when executed by at least one of the one or more processors, cause the system to:
      a. determine first-scale screening conditions based at least in part upon contribution of second-scale conditions to performance parameters of first strains of an organism at the second scale, wherein the first-scale screening conditions include one or more proxies for second-scale conditions that cannot be replicated at first scale;
      b. determine first-scale screening parameters based at least in part upon computer modeling of the metabolism of the organism at the second scale; and
      c. design experiments for experimentally screening second strains of the organism under the first-scale screening conditions based at least in part upon the first-scale screening parameters.
2. The system of embodiment 1, wherein the one or more memories store further instructions that, when executed, generate a first-scale statistical model of first-scale performance of the second strains, and use the first-scale statistical model to predict performance of the second strains at a third scale.
3. The system of embodiment 2, wherein the third scale is the same as the second scale.
4. The system of any one of embodiments 2 or 3, wherein designing experiments includes screening the second strains based at least in part upon the predicted third-scale performance of the second strains.
5. The system of any one of the preceding embodiments, wherein determining first-scale screening conditions is further based at least in part upon environmental conditions determined from fermentation modeling.
6. The system of any one of the preceding embodiments, wherein determining first-scale screening conditions is further based at least in part upon environmental conditions determined from fermentation modeling of the organism at a third scale larger than the second scale.
7. The system of any one of the preceding embodiments, wherein the first scale is at the scale of a plate, and the second scale is at the scale of a bench tank.
8. The system of any one of the preceding embodiments, wherein the first scale is at the scale of a plate comprising wells wherein each well has a volume within a range of 50-200 microliters, and the second scale is at the scale of a bench tank has a volume within a range of 200 ml-10 liters.
9. The system of any one of the preceding embodiments, wherein determining first-scale screening parameters comprises determining second-scale performance parameters that contribute to a key performance indicator (“KPI”) above a contribution threshold.
10. The system of any one of the preceding embodiments, wherein determining first-scale screening parameters comprises determining second-scale performance parameters based on their potential for improving performance of a KPI.
11. The system of any one of the preceding embodiments, wherein the one or more memories store further instructions that, when executed, determine optimum values of the first-scale screening conditions that optimize the first-scale screening parameters collectively at the first scale, wherein designing experiments comprises designing experiments to experimentally determine first-scale performance of the second strains in response to a range of screening condition values around the optimum screening condition values.
12. The system of any one of the preceding embodiments, wherein the one or more memories store further instructions that, when executed, determine optimum values of the first-scale screening conditions that optimize the first-scale screening parameters and a plate-tank deviance collectively at the second scale, wherein designing experiments comprises designing experiments to experimentally determine first-scale performance of the second strains in response to a range of screening condition values around the optimum condition values.
13. The system of any one of the preceding embodiments, wherein the one or more memories store further instructions that, when executed, control performance of experiments to screen the second strains at the first scale using the first-scale screening conditions and the first scale screening parameters.
14. The system of any one of the preceding embodiments, wherein the first strains and the second strains are the same.

### Computer-Readable Medium Embodiments

Set 1
1. One or more non-transitory computer-readable media storing instructions for designing experiments for organisms at a first scale to generate first-scale performance data used in predicting performance of the organisms at a larger, second scale, wherein the instructions, when executed by one or more computing devices, cause at least one of the one or more computing devices to:
   a. determine first-scale screening conditions based at least in part upon contribution of second-scale conditions to performance parameters of first strains of an organism at the second scale, wherein the first-scale screening conditions include one or more proxies for second-scale conditions that cannot be replicated at first scale;
   b. determine first-scale screening parameters based at least in part upon computer modeling of the metabolism of the organism at the second scale; and
   c. design experiments for experimentally screening second strains of the organism under the first-scale screening conditions based at least in part upon the first-scale screening parameters.
2. The computer-readable media of embodiment 1, wherein the computer-readable media store further instructions that, when executed, generate a first-scale statistical model of first-scale performance of the second strains, and use the first-scale statistical model to predict performance of the second strains at a third scale.
3. The computer-readable media of embodiment 2, wherein the third scale is the same as the second scale.
4. The computer-readable media of any one of the embodiments 2 or 3, wherein designing experiments includes screening the second strains based at least in part upon the predicted third-scale performance of the second strains.
5. The computer-readable media of any one of the preceding embodiments, wherein determining first-scale screening conditions is further based at least in part upon environmental conditions determined from fermentation modeling.
6. The computer-readable media of any one of the preceding embodiments, wherein determining first-scale screening conditions is further based at least in part upon environmental conditions determined from fermentation modeling of the organism at a third scale larger than the second scale.
7. The computer-readable media of any one of the preceding embodiments, wherein the first scale is at the scale of a plate, and the second scale is at the scale of a bench tank.
8. The computer-readable media of any one of the preceding embodiments, wherein the first scale is at the scale of a plate comprising wells wherein each well has a volume within a range of 50-200 microliters, and the second scale is at the scale of a bench tank has a volume within a range of 200 ml-10 liters.
9. The computer-readable media of any one of the preceding embodiments, wherein determining first-scale screening parameters comprises determining second-scale performance parameters that contribute to a key performance indicator (“KPI”) above a contribution threshold.
10. The computer-readable media of any one of the preceding embodiments, wherein determining first-scale screening parameters comprises determining second-scale performance parameters based on their potential for improving performance of a KPI.
11. The computer-readable media of any one of the preceding embodiments, wherein the computer-readable media store further instructions that, when executed, determine optimum values of the first-scale screening conditions that optimize the first-scale screening parameters collectively at the first scale, wherein designing experiments comprises designing experiments to experimentally determine first-scale performance of the second strains in response to a range of screening condition values around the optimum screening condition values.
12. The computer-readable media of any one of the preceding embodiments, wherein the computer-readable media store further instructions that, when executed, determine optimum values of the first-scale screening conditions that optimize the first-scale screening parameters and a plate-tank deviance collectively at the second scale, wherein designing experiments comprises designing experiments to experimentally determine first-scale performance of the second strains in response to a range of screening condition values around the optimum condition values.
13. The computer-readable media of any one of the preceding embodiments, wherein the computer-readable media store further instructions that, when executed, control performance of experiments to screen the second strains at the first scale using the first-scale screening conditions and the first scale screening parameters.
14. The computer-readable media of any one of the preceding embodiments, wherein the first strains and the second strains are the same.

1. A computer-implemented method of designing experiments for organisms at a first scale to generate first-scale performance data used in predicting performance of the organisms at a larger, second scale, the method comprising:
a. determining first-scale screening conditions based at least in part upon contribution of second-scale conditions to performance parameters of first strains of an organism at the second scale, wherein the first-scale screening conditions include one or more proxies for second-scale conditions that cannot be replicated at first scale;
b. determining first-scale screening parameters based at least in part upon computer modeling of the metabolism of the organism at the second scale; and
c. designing experiments for experimentally screening second strains of the organism under the first-scale screening conditions based at least in part upon the first-scale screening parameters.

2. The method of claim 1, further comprising generating a first-scale statistical model of first-scale performance of the second strains, and using the first-scale statistical model to predict performance of the second strains at a third scale.

3. The method of claim 2, wherein the third scale is the same as the second scale.

4. The method of claim 2, wherein designing experiments includes screening the second strains based at least in part upon the predicted third-scale performance of the second strains.

5. The method of claim 1, wherein determining first-scale screening conditions is further based at least in part upon environmental conditions determined from fermentation modeling.

6. The method of claim 1, wherein determining first-scale screening conditions is further based at least in part upon environmental conditions determined from fermentation modeling of the organism at a third scale larger than the second scale.

7. The method of claim 1, wherein the first scale is at the scale of a plate, and the second scale is at the scale of a bench tank.

8. The method of claim 1, wherein the first scale is at the scale of a plate comprising wells wherein each well has a volume within a range of 50-200 microliters, and the second scale is at the scale of a bench tank has a volume within a range of 200 ml-10 liters.

9. The method of claim 1, wherein determining first-scale screening parameters comprises determining second-scale performance parameters that contribute to a key performance indicator (“KPI”) above a contribution threshold.

10. The method of claim 1, wherein determining first-scale screening parameters comprises determining second-scale performance parameters based on their potential for improving performance of a KPI.

11. The method of claim 1, further comprising determining optimum values of the first-scale screening conditions that optimize the first-scale screening parameters collectively at the first scale, wherein designing experiments comprises designing experiments to experimentally determine first-scale performance of the second strains in response to a range of screening condition values around the optimum screening condition values.

12. The method of claim 1, further comprising determining optimum values of the first-scale screening conditions that optimize the first-scale screening parameters and a plate-tank deviance collectively at the second scale, wherein designing experiments comprises designing experiments to experimentally determine first-scale performance of the second strains in response to a range of screening condition values around the optimum condition values.

13. The method of claim 1, further comprising controlling performance of experiments to screen the second strains at the first scale using the first-scale screening conditions and the first scale screening parameters.

14. The method of claim 1, wherein the first strains and the second strains are the same.

15. A system for designing experiments for organisms at a first scale to generate first-scale performance data used in predicting performance of the organisms at a larger, second scale, the system comprising:
one or more memories storing instructions; and
one or more processors, operatively coupled to the one or more memories, for executing the instructions to cause the system to:
a. determine first-scale screening conditions based at least in part upon contribution of second-scale conditions to performance parameters of first strains of an organism at the second scale, wherein the first-scale screening conditions include one or more proxies for second-scale conditions that cannot be replicated at first scale;
b. determine first-scale screening parameters based at least in part upon computer modeling of the metabolism of the organism at the second scale; and
c. design experiments for experimentally screening second strains of the organism under the first-scale screening conditions based at least in part upon the first-scale screening parameters.

16. The system of claim 15, wherein the one or more memories store further instructions that, when executed, cause the system to generate a first-scale statistical model of first-scale performance of the second strains, and use the first-scale statistical model to predict performance of the second strains at a third scale.

17. The system of claim 16, wherein the third scale is the same as the second scale.

18. The system of claim 16, wherein designing experiments includes screening the second strains based at least in part upon the predicted third-scale performance of the second strains.

19. The system of claim 15, wherein determining first-scale screening conditions is further based at least in part upon environmental conditions determined from fermentation modeling.

20. The system of claim 15, wherein determining first-scale screening conditions is further based at least in part upon environmental conditions determined from fermentation modeling of the organism at a third scale larger than the second scale.

21. The system of claim 15, wherein the first scale is at the scale of a plate, and the second scale is at the scale of a bench tank.

22. The system of claim 15, wherein the first scale is at the scale of a plate comprising wells wherein each well has a volume within a range of 50-200 microliters, and the second scale is at the scale of a bench tank has a volume within a range of 200 ml-10 liters.

23. The system of claim 15, wherein determining first-scale screening parameters comprises determining second-scale performance parameters that contribute to a key performance indicator (“KPI”) above a contribution threshold.

24. The system of claim 15, wherein determining first-scale screening parameters comprises determining second-scale performance parameters based on their potential for improving performance of a KPI.

25. The system of claim 15, wherein the one or more memories store further instructions that, when executed, cause the system to determine optimum values of the first-scale screening conditions that optimize the first-scale screening parameters collectively at the first scale, wherein designing experiments comprises designing experiments to experimentally determine first-scale performance of the second strains in response to a range of screening condition values around the optimum screening condition values.

26. The system of claim 15, wherein the one or more memories store further instructions that, when executed, cause the system to determine optimum values of the first-scale screening conditions that optimize the first-scale screening parameters and a plate-tank deviance collectively at the second scale, wherein designing experiments comprises designing experiments to experimentally determine first-scale performance of the second strains in response to a range of screening condition values around the optimum condition values.

27. The system of claim 15, wherein the one or more memories store further instructions that, when executed, cause the system to control performance of experiments to screen the second strains at the first scale using the first-scale screening conditions and the first scale screening parameters.

28. The system of claim 15, wherein the first strains and the second strains are the same.

29. One or more non-transitory computer-readable media storing instructions for designing experiments for organisms at a first scale to generate first-scale performance data used in predicting performance of the organisms at a larger, second scale, wherein the instructions, when executed by one or more computing devices, cause at least one of the one or more computing devices to:
a. determine first-scale screening conditions based at least in part upon contribution of second-scale conditions to performance parameters of first strains of an organism at the second scale, wherein the first-scale screening conditions include one or more proxies for second-scale conditions that cannot be replicated at first scale;
b. determine first-scale screening parameters based at least in part upon computer modeling of the metabolism of the organism at the second scale; and
c. design experiments for experimentally screening second strains of the organism under the first-scale screening conditions based at least in part upon the first-scale screening parameters.

30. The one or more computer-readable media of claim 29, wherein the computer-readable media store further instructions that, when executed, cause at least one of the one or more computing devices to generate a first-scale statistical model of first-scale performance of the second strains, and use the first-scale statistical model to predict performance of the second strains at a third scale.

31. The one or more computer-readable media of claim 30, wherein the third scale is the same as the second scale.

32. The one or more computer-readable media of claim 29, wherein designing experiments includes screening the second strains based at least in part upon the predicted third-scale performance of the second strains.

33. The one or more computer-readable media of claim 29, wherein determining first-scale screening conditions is further based at least in part upon environmental conditions determined from fermentation modeling.

34. The one or more computer-readable media of claim 29, wherein determining first-scale screening conditions is further based at least in part upon environmental conditions determined from fermentation modeling of the organism at a third scale larger than the second scale.

35. The one or more computer-readable media of claim 29, wherein the first scale is at the scale of a plate, and the second scale is at the scale of a bench tank.

36. The one or more computer-readable media of claim 29, wherein the first scale is at the scale of a plate comprising wells wherein each well has a volume within a range of 50-200 microliters, and the second scale is at the scale of a bench tank has a volume within a range of 200 ml-10 liters.

37. The one or more computer-readable media of claim 29, wherein determining first-scale screening parameters comprises determining second-scale performance parameters that contribute to a key performance indicator (“KPI”) above a contribution threshold.

38. The one or more computer-readable media of claim 29, wherein determining first-scale screening parameters comprises determining second-scale performance parameters based on their potential for improving performance of a KPI.

39. The one or more computer-readable media of claim 29, wherein the computer-readable media store further instructions that, when executed, cause at least one of the one or more computing devices to determine optimum values of the first-scale screening conditions that optimize the first-scale screening parameters collectively at the first scale, wherein designing experiments comprises designing experiments to experimentally determine first-scale performance of the second strains in response to a range of screening condition values around the optimum screening condition values.

40. The one or more computer-readable media of claim 29, wherein the computer-readable media store further instructions that, when executed, cause at least one of the one or more computing devices to determine optimum values of the first-scale screening conditions that optimize the first-scale screening parameters and a plate-tank deviance collectively at the second scale, wherein designing experiments comprises designing experiments to experimentally determine first-scale performance of the second strains in response to a range of screening condition values around the optimum condition values.

41. The one or more computer-readable media of claim 29, wherein the computer-readable media store further instructions that, when executed, cause at least one of the one or more computing devices to control performance of experiments to screen the second strains at the first scale using the first-scale screening conditions and the first scale screening parameters.

42. The one or more computer-readable media of claim 29, wherein the first strains and the second strains are the same.
